(19) United States
(12) Patent Application Publication
(10) Pub. No.: US 2026/0049068 A1
(43) Pub. Date: Feb. 19, 2026

(54) Substituted Tetrazolyl Compounds Useful as T Cell Activators

(71) Applicant: BRISTOL-MYERS SQUIBB COMPANY, Princeton, NJ (US)

(72) Inventors: Xiaofan Zheng, Cheshire, CT (US); Louis S. Chupak, Old Saybrook, CT (US); Upender Velaparthi, Princeton Junction, NJ (US); Jayakumar Sankara Warrier, Bangalore (IN)

(21) Appl. No.: 19/101,628

(22) PCT Filed: Aug. 7, 2023
(86) PCT No.: PCT/US2023/071761

§ 371 Date: Feb. 6, 2025

Related U.S. Application Data
(60) Provisional application No. 63/370,734, filed on Aug. 8, 2022.

(51) Int. Cl. C07D 401/04; A61K 31/41; A61K 31/4439; C07D 257/04; C07D 403/06; C07D 403/12; C07D 405/12

(52) U.S. Cl. CPC C07D 401/04; A61K 31/41; A61K 31/4439; C07D 257/04; C07D 403/06; C07D 403/12; C07D 405/12

(57) ABSTRACT

Disclosed are compounds of Formula (I): or a salt thereof, wherein: X, Y, Z, R₁, R₂, R₃, and n are defined herein. Also disclosed are methods of using such compounds to inhibit the activity of one or both of diacylglycerol kinase alpha (DGKα) and diacylglycerol kinase zeta (DGKζ), and pharmaceutical compositions comprising such compounds. These compounds are useful in the treatment of viral infections and proliferative disorders, such as cancer.

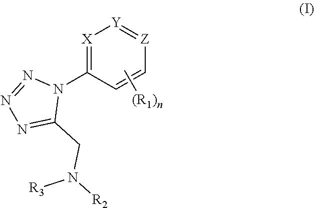

### CROSS REFERENCE

This application claims the benefit of U.S. Provisional Application No. 63/370,734 filed Aug. 8, 2022 which is incorporated herein in its entirety.

### DESCRIPTION

The present invention generally relates to substituted tetrazolyl compounds that activate T cells, promote T cell proliferation, and/or exhibit antitumor activity. Provided herein are substituted tetrazolyl compounds, compositions comprising such compounds, and methods of their use. The invention further pertains to pharmaceutical compositions comprising at least one compound according to the invention that are useful for the treatment of proliferative disorders, such as cancer, and viral infections.

### REFERENCE TO SEQUENCE LISTING SUBMITTED ELECTRONICALLY VIA EFS-WEB

Incorporated herein by reference in its entirety is a Sequence Listing entitled, “14166WOPCT” comprising SEQ ID NO: 1 through SEQ ID NO: 6, which include nucleic acid and/or amino acid sequences disclosed herein. The Sequence Listing has been submitted herein in XML format via Patent Center, and thus constitutes both the paper and computer readable form thereof. The Sequence Listing was first created using WIPO Sequence on Jul. 25, 2023, and is 16.0 KB.

### BACKGROUND OF THE INVENTION

Human cancers harbor numerous genetic and epigenetic alterations, generating neoantigens potentially recognizable by the immune system (Sjoblom et al. (2006) Science 314:268-74). The adaptive immune system, comprised of T and B lymphocytes, has powerful anti-cancer potential, with a broad capacity and exquisite specificity to respond to diverse tumor antigens. Further, the immune system demonstrates considerable plasticity and a memory component. The successful harnessing of all these attributes of the adaptive immune system would make immunotherapy unique among all cancer treatment modalities. However, although an endogenous immune response to cancer is observed in preclinical models and patients, this response is ineffective, and established cancers are viewed as “self” and tolerated by the immune system. Contributing to this state of tolerance, tumors may exploit several distinct mechanisms to actively subvert anti-tumor immunity. These mechanisms include dysfunctional T-cell signaling (Mizoguchi et al., (1992) Science 258:1795-98), suppressive regulatory cells (Facciabene et al., (2012) Cancer Res. 72:2162-71), and the co-opting of endogenous “immune checkpoints”, which serve to down-modulate the intensity of adaptive immune responses and protect normal tissues from collateral damage, by tumors to evade immune destruction (Topalian et al., (2012) Curr. Opin. Immunol. 24:1-6; Mellman et al. (2011) Nature 480:480-489).

Diacylglycerol kinases (DGKs) are lipid kinases that mediate the conversion of diacylglycerol to phosphatidic acid thereby terminating T cell functions propagated through the TCR signaling pathway. Thus. DGKs serve as intracellular checkpoints and inhibition of DGKs are expected to enhance T cell signaling pathways and T cell activation. Supporting evidence include knock-out mouse models of either DGKα or DGKζ which show a hyper-responsive T cell phenotype and improved anti-tumor immune activity (Riese M. J. et al., Journal of Biological Chemistry, (2011) 7: 5254-5265; Zha Y et al., Nature Immunology, (2006) 12:1343, Olenchock B. A. et al., (2006) 11: 1174-81). Furthermore tumor infiltrating lymphocytes isolated from human renal cell carcinoma patients were observed to overexpress DGKα which resulted in inhibited T cell function (Prinz, P. U. et al., J Immunology (2012) 12:5990-6000). Thus, DGKα and DGKζ are viewed as targets for cancer immunotherapy (Riese M. J. et al., Front Cell Dev Biol. (2016) 4: 108, Chen, S. S. et al., Front Cell Dev Biol. (2016) 4: 130; Avila-Flores, A. et al., Immunology and Cell Biology (2017) 95: 549-563: Noessner, E., Front Cell Dev Biol. (2017) 5: 16; Krishna, S., et al., Front Immunology (2013) 4:178; Jing, W. et al., Cancer Research (2017) 77: 5676-5686.

There remains a need for compounds useful as inhibitors of one or both of DGKα and DGKζ. Additionally, there remains a need for compounds useful as inhibitors of one or both of DGKα and DGKζ that have selectivity over other diacylglycerol kinases, protein kinases, and/or other lipid kinases.

Accordingly, an agent that is safe and effective in restoring T cell activation, lowering antigen threshold, enhancing antitumor functionality, and/or overcoming the suppressive effects of one or more endogenous immune checkpoints, such as PD-1, LAG-3 and TGFβ, would be an important addition for the treatment of patients with proliferative disorders, such as cancer, as well as viral infections.

### SUMMARY OF THE INVENTION

Applicants have found compounds that have activity as inhibitors of one or both of DGKα and DGKζ. Further, applicants have found compounds that have activity as inhibitors of one or both of DGKα and DGKζ and have selectivity over other diacylglycerol kinases, protein kinases, and/or other lipid kinases. These compounds are provided to be useful as pharmaceuticals with desirable stability, bioavailability, therapeutic index, and toxicity values that are important to their drugability.

The present invention provides substituted tetrazolyl compounds of Formula (I), which are useful as inhibitors of DGKα, DGKζ, or both DGKα and DGKζ, including salts and prodrugs thereof.

The present invention also provides pharmaceutical compositions comprising a compound of Formula (I) and/or a pharmaceutically acceptable salt thereof; and a pharmaceutically acceptable carrier.

The present invention also provides a method of treating a disease or disorder associated with the activity of DGKα, DGKζ, or both DGKα and DGKζ, the method comprising administering to a mammalian patient a compound of Formula (I) and/or a pharmaceutically acceptable salt thereof.

The present invention also provides processes and intermediates for making the compounds of Formula (I) and/or salts thereof.

The present invention also provides a compound of Formula (I) and/or a pharmaccutically acceptable salt thereof, for use in therapy.

The present invention also provides the use of the compounds of Formula (I) and/or pharmaceutically acceptable salts thereof, for the manufacture of a medicament for the treatment of proliferative disorders, such as cancer and viral infections.

The compounds of Formula (I) and compositions comprising the compounds of Formula (I) may be used in treating, preventing, or curing viral infections and various proliferative disorders, such as cancer. Pharmaceutical compositions comprising these compounds are useful in treating, preventing, or slowing the progression of diseases or disorders in a variety of therapeutic areas, such as viral infections and cancer.

These and other features of the invention will be set forth in expanded form as the disclosure continues.

### DETAILED DESCRIPTION

The first aspect of the present invention provides at least one compound of Formula (I):

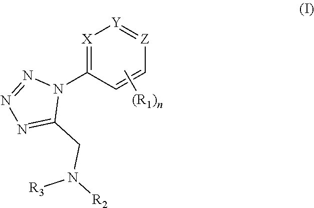

or a salt thereof, wherein:
(i) X is N, CH, or CR₁; Y is N, CH, or CR₁; and Z is CH or CR₁; provided that zero or one of X and Y is N; or
(ii) X is CH or CR₁; Y is NR₁ₐ; and Z is C(═O);
═ represents either a single bond when Z is C(═O) or a double bond when Z is CH or CR₁;
each R₁ is independently F, Cl, Br, —CN, C₁₋₃ alkyl, C₁₋₂ fluoroalkyl, C₁₋₃ alkoxy, C₁₋₂ fluoroalkoxy, —C(O)OH, —C(O)O(C₁₋₃ alkyl), or —NO₂;
R₁ₐ is hydrogen or —CH₃;
R₂ is C₃₋₄ alkyl or a cyclic group selected from C₃₋₆ cycloalkyl, oxetanyl, tetrahydrofuranyl, tetrahydropyranyl, azetidinyl, pyrrolidinyl, piperidinyl, piperazinyl, bicyclo[2.2.1]heptanyl, bicyclo[3.1.0]hexanyl, bicyclo[4.1.0]heptanyl, bicyclo[3.1.1]heptanyl, bicyclo[3.2.1]octanyl, bicyclo[2.2.2]octanyl, spiro[2.5]octanyl, cubanyl, phenyl, and a 5- to 6-membered heteroaryl having 1 to 3 heteroatoms selected from N, O, and S, wherein each cyclic group is substituted with zero to 3 R₂ₐ;
each R₂ₐ is independently F, Cl, Br, —OH, —CN, C₁₋₃ alkyl, C₁₋₂ fluoroalkyl, or —C(O)O(C₁₋₂ alkyl);
R₃ is C₁₋₆ alkyl, C₁₋₃ fluoroalkyl, C₁₋₄ hydroxyalkyl, C₃₋₆ cycloalkyl, —CH₂(C₃₋₆ cycloalkyl), —CH₂(phenyl), —CRₓRₓCRₓ(OH)(phenyl), —CRₓRₓCRₓ=CRₓRₓ, —(CRₓRₓ)₁₋₂C(O)O(C₁₋₂ alkyl), or —(CRₓRₓ)₁₋₃NRₓC(O)(phenyl);
or alternatively, R₂ and R₃ along with the nitrogen atom to which they are attached form pyrrolidinyl or piperidinyl, each substituted with zero to 2 R₃ₐ;
each R₃ₐ is independently F, Cl, —CN, —OH, C₁₋₃ alkyl, or C₁₋₃ fluoroalkyl;
each Rₓ is independently hydrogen or —CH₃; and
n is zero, 1, 2, or 3;
with the provisos:
(i) the moiety

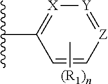

is not

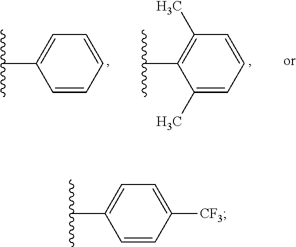

and
(ii) wherein the compound of Formula (I) is not

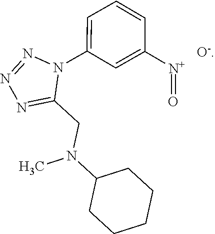

The embodiments hereinbelow include proviso (i) and proviso (ii) of the first aspect.

In one embodiment, a compound of Formula (I) or a salt thereof is provided wherein X is N, CH, or CR₁; Y is N, CH, or CR₁; Z is CH or CR₁; and ═ represents a double bond; provided that zero or one of X and Y is N. Compounds of this embodiment have the structure of Formula (II):

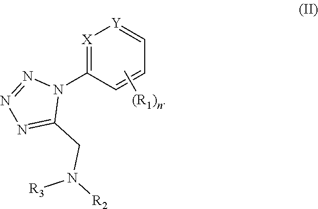

In one embodiment, a compound of Formula (I) or a salt thereof is provided wherein X is CH or CR₁; Y is CH or CR₁; Z is CH or CR₁; and ═ represents a double bond. Compounds of this embodiment have the structure of Formula (IIa):

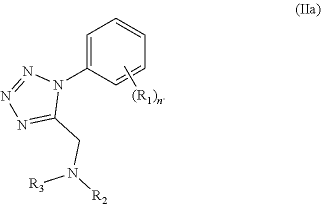

In one embodiment, a compound of Formula (I) or a salt thereof is provided wherein X is N; Y is CH or CR₁; Z is CH or CR₁; and =represents a double bond. Compounds of this embodiment have the structure of Formula (IIb):

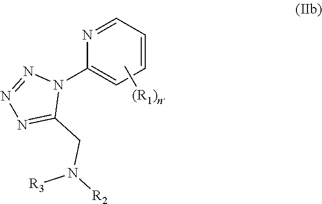

In one embodiment, a compound of Formula (I) or a salt thereof is provided wherein X is CH or CR₁; Y is N; Z is CH or CR₁; and =represents a double bond. Compounds of this embodiment have the structure of Formula (IIc):

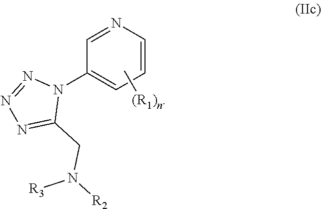

In one embodiment, a compound of Formula (I) or a salt thereof is provided wherein X is N, CH, or CR₁; Y is N, CH, or CR₁; Z is CH or CR₁; ═ represents a double bond; and one of X and Y is N.

In one embodiment, a compound of Formula (I) or a salt thereof is provided wherein X is CH or CR₁; Y is NR₁ₐ; Z is C(═O); and =represents either a single bond. Compounds of this embodiment have the structure of Formula (III):

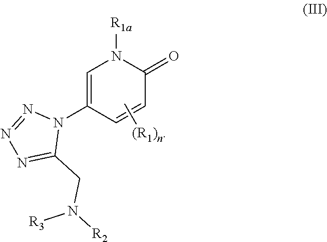

In one embodiment, a compound of Formula (I) or a salt thereof is provided wherein (i) X is N, CH, or CR₁; Y is N, CH, or CR₁; and Z is CH or CR₁; provided that zero or one of X and Y is N; or (ii) X is CH or CR₁; Y is NR₁ₐ; and Z is C(═O); ═ represents either a single bond when Z is C(═O) or a double bond when Z is CH or CR₁; each R₁ is independently F, Cl, Br, —CN, C₁₋₃ alkyl, C₁₋₂ fluoroalkyl, C₁₋₃ alkoxy, C₁₋₂ fluoroalkoxy, —C(O)OH, —C(O)O(C₁₋₂ alkyl), or —NO₂; R₁ₐ is hydrogen or —CH₃; R₂ is C₃₋₄ alkyl or a cyclic group selected from C₃₋₆ cycloalkyl, oxetanyl, tetrahydrofuranyl, tetrahydropyranyl, azetidinyl, pyrrolidinyl, piperidinyl, piperazinyl, bicyclo[2.2.1]heptanyl, bicyclo[3.1.0]hexanyl, bicyclo[4.1.0]heptanyl, bicyclo[3.1.1]heptanyl, bicyclo[3.2.1]octanyl, bicyclo[2.2.2]octanyl, spiro[2.5]octanyl, cubanyl, phenyl, and a 5- to 6-membered heteroaryl having 1 to 3 heteroatoms selected from N, O, and S, wherein each cyclic group is substituted with zero to 3 R₂ₐ; each R₂ₐ is independently F, Cl, Br, —OH, —CN, C₁₋₃ alkyl, C₁₋₂ fluoroalkyl, or —C(O)O(C₁₋₂ alkyl); R₃ is C₁₋₆ alkyl, C₁₋₃ fluoroalkyl, C₁₋₄ hydroxyalkyl, C₃₋₆ cycloalkyl, —CH₂(C₃₋₆ cycloalkyl), —CH₂(phenyl), —CRₓRₓCRₓ(OH)(phenyl), —CRₓRₓCRₓ=CRₓRₓ, —(CRₓRₓ)₁₋₂C(O)O(C₁₋₂ alkyl), or —(CRₓRₓ)₁₋₃NRₓC(O)(phenyl); each Rₓ is independently hydrogen or —CH₃; and n is zero, 1, 2, or 3.

In one embodiment, a compound of Formula (I) or a salt thereof is provided wherein X is N, CH, or CR₁; Y is N, CH, or CR₁; and Z is CH or CR₁; provided that zero or one of X and Y is N, ═ represents a double bond; each R₁ is independently F, Cl, Br, —CN, C₁₋₃ alkyl, C₁₋₂ fluoroalkyl, C₁₋₃ alkoxy, C₁₋₂ fluoroalkoxy, —C(O)OH, —C(O)O(C₁₋₃ alkyl), or —NO₂; R₁ₐ is hydrogen or —CH₃; R₂ is C₃₋₄ alkyl or a cyclic group selected from C₃₋₆ cycloalkyl, oxetanyl, tetrahydrofuranyl, tetrahydropyranyl, azetidinyl, pyrrolidinyl, piperidinyl, piperazinyl, bicyclo[2.2.1]heptanyl, bicyclo[3.1.0]hexanyl, bicyclo[4.1.0]heptanyl, bicyclo[3.1.1]heptanyl, bicyclo[3.2.1]octanyl, bicyclo[2.2.2]octanyl, spiro[2.5]octanyl, cubanyl, phenyl, and a 5- to 6-membered heteroaryl having 1 to 3 heteroatoms selected from N, O, and S, wherein each cyclic group is substituted with zero to 3 R₂ₐ; each R₂ₐ is independently F, Cl, Br, —OH, —CN, C₁₋₃ alkyl, C₁₋₂ fluoroalkyl, or —C(O)O(C₁₋₂ alkyl); R₃ is C₁₋₆ alkyl, C₁₋₃ fluoroalkyl, C₁₋₄ hydroxyalkyl, C₃₋₆ cycloalkyl. —CH₂(C₃₋₆ cycloalkyl), —CH₂(phenyl), —CRₓRₓCRₓ(OH)(phenyl), —CRₓRₓCRₓ═CRₓRₓ, —(CRₓRₓ)₁₋₂C(O)O(C₁₋₂ alkyl), or —(CRₓRₓ)₁₋₃NRₓC(O)(phenyl); or alternatively, R₂ and R₃ along with the nitrogen atom to which they are attached form pyrrolidinyl or piperidinyl, each substituted with zero to 2 R₃ₐ; each R₃ₐ is independently F, Cl, —CN, —OH, C₁₋₃ alkyl, or C₁₋₃ fluoroalkyl; and each Rₓ is independently hydrogen or —CH₃; and n is zero, 1, 2, or 3.

In one embodiment, a compound of Formula (I) or a salt thereof is provided wherein: each R₁ is independently F, Cl, Br, —CN, C₁₋₃ alkyl, —CHF₂, —CF₃, —OCH₃, —OCF₃, —C(O)OH, —C(O)O(C₁₋₂ alkyl), or —NO₂; R₂ is C₃₋₄ alkyl or a cyclic group selected from C₃₋₆ cycloalkyl, tetrahydrofuranyl, tetrahydropyranyl, pyrrolidinyl, and phenyl, each substituted with zero to 2 R₂ₐ; each R₂ₐ is independently —OH, —CN, —CH₃, —CH₂F, —CHF₂, —CF₃, or —C(O)OCH₂CH₃; R₃ is C₁₋₄ alkyl, C₁₋₂ fluoroalkyl, CH₃ hydroxyalkyl, C₃₋₄ cycloalkyl, —CH₂(C₃₋₄ cycloalkyl), —CH₂(phenyl), —CH₂CH(OH)(phenyl), —CH₂CH═CH₂, —CH₂CH₂C(O)O(C₁₋₂ alkyl), or —CH₂CH₂CH₂NHC(O)(phenyl); or alternatively, R₂ and R₃ along with the nitrogen atom to which they are attached form pyrrolidinyl or piperidinyl, each substituted with zero to 2 R₃ₐ each R₃ₐ is independently —CN, —OH, —CH₃, or —CF₃; and n is zero, 1, 2, or 3.

In one embodiment, a compound of Formula (I) or a salt thereof is provided wherein: each R₁ is independently F, Cl, Br, —CN, C₁₋₃ alkyl, —CHF₂, —CF₃, —OCH₃, —OCF₃, —C(O)OH, —C(O)OCH₃, or —NO—; R₂ is C₃₋₄ alkyl or a cyclic group selected from C₃₋₆ cycloalkyl, tetrahydrofuranyl, tetrahydropyranyl, pyrrolidinyl, and phenyl, each substituted with zero to 2 R₂ₐ, each R₂ₐ is independently —OH, —CN, —CH₃, —CH₂F, —CHF₂, —CF₃, or —C(O)OCH₂CH₃; and R₁ is C₁₋₄ alkyl, C₁₋₂ fluoroalkyl, C₁₋₃ hydroxyalkyl, C₃₋₄ cycloalkyl, —CH₂(C₃₋₄ cycloalkyl), —CH₂(phenyl), —CH₂CH(OH)(phenyl), —CH₂CH═CH₂, —CH₂CH₂C(O)O(C₁₋₂ alkyl), or —CH₂CH₂CH₂NHC(O)(phenyl).

In one embodiment, a compound of Formula (I) or a salt thereof is provided wherein each R₁ is independently F, Cl, Br, —CN, —CH₃, —CF₃, —OCH₃, —C(O)OH, —C(O)OCH₃, or —NO₂; R₂ is —CH(CH₃)₂, —CH₂CH(CH₃)₂, cyclopropyl, cyclohexyl substituted with zero to 2 R₂ₐ, tetrahydrofuranyl, tetrahydropyranyl, pyrrolidinyl substituted with —C(O)OCH₂CH₃, or phenyl; each R₂ₐ is independently —OH, —CH₃, or —C(O)OCH₂CH₃; R₃ is —CH₃, —CH₂CH₃, —CH₂CH₂CH₃, —CH(CH₃)₂, —CH₂CH₂CH₂CH₃, —CH₂CH₂OH, cyclopropyl, —CH₂(cyclopropyl), —CH₂(phenyl), —CH₂CH(OH)(phenyl), —CH₂CH═CH₂, —CH₂CH₂C(O)OCH₃, or —CH₂CH₂CH₂NHC(O)(phenyl); or alternatively, R₂ and R₃ along with the nitrogen atom to which they are attached form pyrrolidinyl substituted with 2 R₃ₐ; each R₃ₐ is —CH₃; and n is 1 or 2.

In one embodiment, a compound of Formula (I) or a salt thereof is provided wherein each R₁ is independently F, Cl, Br, —CN, C₁₋₃ alkyl, C₁₋₂ fluoroalkyl, C₁₋₂alkoxy, C₁₋₂ fluoroalkoxy, —C(O)OH, —C(O)O(C₁₋₃ alkyl), or —NO₂. Included in this embodiment are compounds in which each R₁ is independently F, Cl, Br, —CN, C₁₋₃ alkyl, —CHF₂, —CF₃, —OCH₃, —OCF₃, or —NO₂. Also included in this embodiment are compounds in which each R₁ is independently F, Cl, Br, —CN, —CH₃, —CF₃, —OCH₃, —C(O)OH, —C(O)OCH₃, or —NO₂.

In one embodiment, a compound of Formula (I) or a salt thereof is provided wherein R₂ is C₃₋₄ alkyl or a cyclic group selected from C₃₋₆ cycloalkyl, oxetanyl, tetrahydrofuranyl, tetrahydropyranyl, azetidinyl, pyrrolidinyl, piperidinyl, piperazinyl, bicyclo[2.2.1]heptanyl, bicyclo[3.1.0]hexanyl, bicyclo[4.1.0]heptanyl, bicyclo[3.1.1]heptanyl, bicyclo[3.2.1]octanyl, bicyclo[2.2.2]octanyl, spiro[2.5]octanyl, cubanyl, phenyl, and a 5- to 6-membered heteroaryl having 1 to 3 heteroatoms selected from N, O, and S, wherein each cyclic group is substituted with zero to 2 R₂ₐ. Included in this embodiment are compounds in which R₂ is C₃₋₄ alkyl or a cyclic group selected from C₃₋₆ cycloalkyl, tetrahydrofuranyl, tetrahydropyranyl, pyrrolidinyl, and phenyl, each substituted with zero to 2 R₂ₐ. Also included in this embodiment are compounds in which R₂ is —CH(CH₃)₂, —CH₂CH(CH₃)₂, cyclopropyl, cyclohexyl substituted with zero to 2 R₂ₐ, tetrahydrofuranyl, tetrahydropyranyl, pyrrolidinyl substituted with —C(O)OCH₂CH₃, or phenyl.

In one embodiment, a compound of Formula (I) or a salt thereof is provided wherein R₂ is C₃₋₄ alkyl. Included in this embodiment are compounds in which R₂ is —CH(CH₃)₂ or —CH₂CH(CH₃)₂.

In one embodiment, a compound of Formula (I) or a salt thereof is provided wherein R₂ is a cyclic group selected from C₃₋₆ cycloalkyl, oxetanyl, tetrahydrofuranyl, tetrahydropyranyl, azetidinyl, pyrrolidinyl, piperidinyl, piperazinyl, bicyclo[2.2.1]heptanyl, bicyclo[3.1.0]hexanyl, bicyclo[4.1.0]heptanyl, bicyclo[3.1.1]heptanyl, bicyclo[3.2.1]octanyl, bicyclo[2.2.2]octanyl, spiro[2.5]octanyl, cubanyl, phenyl, and a 5- to 6-membered heteroaryl having 1 to 3 heteroatoms selected from N, O, and S, wherein each cyclic group is substituted with zero to 3 R₂ₐ. Included in this embodiment are compounds in which R₂ is a cyclic group selected from C₃₋₆ cycloalkyl, tetrahydrofuranyl, tetrahydropyranyl, pyrrolidinyl, and phenyl, each substituted with zero to 2 R₂ₐ. Also included in this embodiment are compounds in which R₂ is cyclopropyl, cyclohexyl substituted with zero to 2 R₂ₐ, tetrahydrofuranyl, tetrahydropyranyl, pyrrolidinyl substituted with —C(O)OCH₂CH₃, or phenyl.

In one embodiment, a compound of Formula (I) or a salt thereof is provided wherein R₂ is cyclohexyl.

In one embodiment, a compound of Formula (I) or a salt thereof is provided wherein each R₂ₐ is independently F, Cl, Br, —OH, —CN, C₁₋₂ alkyl, C₁₋₂ fluoroalkyl, or —C(O)O(C₁₋₂ alkyl). Included in this embodiment are compounds in which each R₂ₐ is independently —OH, —CN, —CH₃, —CH₂F, —CHF₂, —CF₃, or —C(O)OCH₂CH₃. Also included in this embodiment are compounds in which each R₂ₐ is independently —OH, —CH₃, or —C(O)OCH₂CH₃.

In one embodiment, a compound of Formula (I) or a salt thereof is provided wherein R₃ is C₁₋₅ alkyl, C₁₋₃ fluoroalkyl, C₁₋₃ hydroxyalkyl, C₃₋₆ cycloalkyl, —CH₂(C₃₋₆ cycloalkyl), —CH₂(phenyl), —CRₓRₓCRₓ(OH)(phenyl), —CRₓRₓ CRₓ═CRₓRₓ, —(CRₓRₓ)₁₋₂C(O)O(C₁₋₂ alkyl), or —(CRₓRₓ)₁₋₃NRₓC(O)(phenyl). Included in this embodiment are compounds in which R₃ is C₁₋₄ alkyl, C₁₋₂ fluoroalkyl, C₁₋₃ hydroxyalkyl, C₃₋₄ cycloalkyl, —CH₂(C₃₋₄ cycloalkyl), —CH₂(phenyl), —CH₂CH(OH)(phenyl), —CH₂CH═CH₂, —CH₂CH₂C(O)O(C₁₋₂ alkyl), or —CH₂CH₂CH₂NHC(O)(phenyl). Also included in this embodiment are compounds in which R₃ is —CH₃, —CH₂CH₃, —CH₂CH₂CH₃, —CH(CH₃)₂, —CH₂CH₂CH₂CH₃, —CH₂CH₂OH, cyclopropyl, —CH₂(cyclopropyl), —CH₂(phenyl), —CH₂CH(OH)(phenyl), —CH₂CH═CH₃, —CH₂CH₂C(O)OCH₃, or —CH₂CH₂CH₂NHC(O)(phenyl).

In one embodiment, a compound of Formula (I) or a salt thereof is provided wherein R₃ is —CH₃.

In one embodiment, a compound of Formula (I) or a salt thereof is provided wherein R₂ is cyclohexyl and R₃ is —CH₃.

In one embodiment, a compound of Formula (I) or a salt thereof is provided wherein R₂ and R₃ along with the nitrogen atom to which they are attached form pyrrolidinyl or piperidinyl, each substituted with zero to 2 R₃ₐ. Included in this embodiment are compounds in which R₂ and R₃ along with the nitrogen atom to which they are attached form pyrrolidinyl substituted with 2 R₃ₐ. Also included in this embodiment are compounds in which R₂ and R₃ along with the nitrogen atom to which they are attached form piperidinyl substituted with 2 R₃ₐ.

In one embodiment, a compound of Formula (I) or a salt thereof is provided wherein each R₃ₐ is independently F, Cl, —CN, —OH, C₁₋₂ alkyl, or C₁₋₂ fluoroalkyl. Included in this embodiment are compounds in which each R₃ₐ is independently —CN, —OH, —CH₃, or —CF₃. Also included in this embodiment are compounds in which each R₃ₐ is —CN or —CH₃. Additionally, included in this embodiment are compounds in which each R₃ₐ is —CH₃.

In one embodiment, a compound of Formula (I) or a salt thereof is provided wherein the compound has the structure selected from:

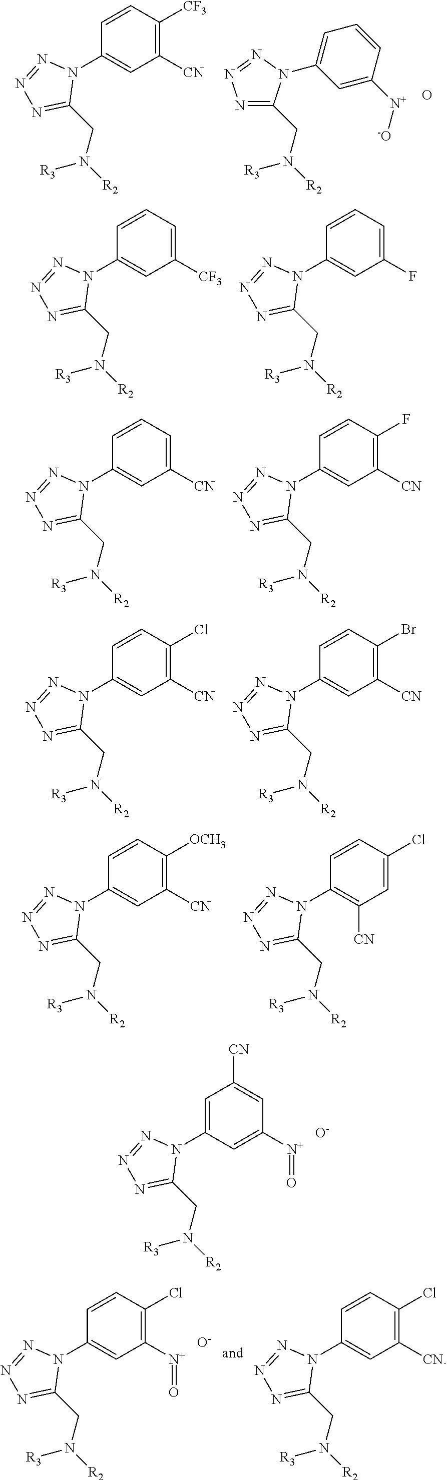

In one embodiment, a compound of Formula (I) or a salt thereof is provided wherein the compound has the structure selected from:

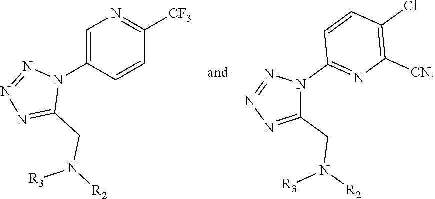

In one embodiment, a compound of Formula (I) or a salt thereof is provided wherein said compound is: methyl 3-(cyclohexyl((1-(3-nitrophenyl)-1H-tetrazol-5-yl) methyl)amino)propanoate (1); N,4-dimethyl-N-((1-(3-nitrophenyl)-1H-tetrazol-5-yl) methyl)cyclohexan-1-amine (2); N-ethyl-N-((1-(3-nitrophenyl)-1H-tetrazol-5-yl)methyl) cyclohexanamine (3); 5-(5-((cyclohexyl(methyl)amino)methyl)-1H-tetrazol-1-yl)-2-(trifluoromethyl)benzonitrile (4); 5-(5-((butyl(cyclohexyl)amino)methyl)-1H-tetrazol-1-yl)-2-chlorobenzonitrile (5); 5-(5-((allyl(cyclohexyl)amino)methyl)-1H-tetrazol-1-yl)-2-chlorobenzonitrile (6); 2-chloro-5-(5-((cyclohexyl(2-hydroxyethyl)amino)methyl)-1H-tetrazol-1-yl)benzonitrile (7); 2-chloro-5-(5-((cyclohexyl(propyl)amino)methyl)-1H-tetrazol-1-yl)benzonitrile (8); 2-chloro-5-(5-((isobutyl(methyl)amino)methyl)-1H-tetrazol-1-yl)benzonitrile (9); 2-chloro-5-(5-((cyclohexyl(cyclopropylmethyl)amino) methyl)-1H-tetrazol-1-yl)benzonitrile (10); 2-chloro-5-(5-((isopropyl(methyl)amino) methyl)-1H-tetrazol-1-yl)benzonitrile (11); 2-chloro-5-(5-((cyclohexyl(isopropyl)amino) methyl)-1H-tetrazol-1-yl)benzonitrile (12); 2-chloro-5-(5-((cyclohexyl(ethyl)amino) methyl)-1H-tetrazol-1-yl)benzonitrile (13); 3-chloro-6-(5-((cyclohexyl(methyl)amino) methyl)-1H-tetrazol-1-yl)picolinonitrile (14); 3-chloro-6-(5-((cyclohexyl (cyclopropylmethyl)amino)methyl)-1H-tetrazol-1-yl) picolinonitrile (15); 3-chloro-6-(5-((cyclohexyl(ethyl)amino)methyl)-1H-tetrazol-1-yl)picolinonitrile (16); 6-(5-((allyl (cyclohexyl)amino)methyl)-1H-tetrazol-1-yl)-3-chloropicolinonitrile (17); N-methyl-N-((1-(3-(trifluoromethyl)phenyl)-1H-tetrazol-5-yl)methyl) cyclohexanamine (18); N-((1-(3-fluorophenyl)-1H-tetrazol-5-yl)methyl)-N-methylcyclohexanamine (19): 3-(5-((cyclohexyl(methyl)amino)methyl)-1H-tetrazol-1-yl)benzonitrile (20): 5-(5-((cyclohexyl (methyl)amino)methyl)-1H-tetrazol-1-yl)-2-fluorobenzonitrile (21); 2-chloro-5-(5-((cyclohexyl(methyl)amino)methyl)-1H-tetrazol-1-yl)benzonitrile (22); N-methyl-N-((1-(6-(trifluormethyl)pyridin-3-yl)-1H-tetrazol-5-yl)methyl) cyclohexanamine (23); 5-(5-((cyclohexyl(methyl)amino)methyl)-1H-tetrazol-1-yl)-2-methoxybenzonitrile (24); 5-(5-((cyclohexyl(methyl)amino)methyl)-1H-tetrazol-1-yl)-2-methylbenzonitrile (25): 2-bromo-5-(5-((cyclohexyl(methyl)amino)methyl)-1H-tetrazol-1-yl)benzonitrile (26); 3-(5-((cyclohexyl(methyl)amino)methyl)-1H-tetrazol-1-yl)-5-nitrobenzonitrile (27); N-((1-(4-chloro-3-nitrophenyl)-1H-tetrazol-5-yl)methyl)-N-methylcyclohexanamine (28); N-((1-(4-chlorophenyl)-1H-tetrazol-5-yl)methyl)-N-methylcyclohexanamine (29): 5-chloro-2-(5-((cyclohexyl(methyl)amino)methyl)-1H-tetrazol-1-yl)benzonitrile (30): N-((1-(4-methoxyphenyl)-1H-tetrazol-5-yl)methyl)-N-methylcyclohexanamine (31); N-methyl-N-((1-(4-nitrophenyl)-1H-tetrazol-5-yl)methyl)cyclohexanamine (32); methyl 3-(5-((cyclohexyl(methyl)amino)methyl)-1H-tetrazol-1-yl)benzoate (33): 3-(5-((cyclohexyl (methyl)amino)methyl)-1H-tetrazol-1-yl)benzoic acid (34); N-methyl-N-((1-(m-tolyl)-1H-tetrazol-5-yl)methyl)cyclohexanamine (35): N-((1-(3-chlorophenyl)-1H-tetrazol-5-yl) methyl)-N-methylcyclohexanamine (36); N-methyl-N-((1-(3-nitrophenyl)-1H-tetrazol-5-yl)methyl)propan-2-amine (37); N-methyl-N-((1-(3-nitrophenyl)-1H-tetrazol-5-yl) methyl) tetrahydro-2H-pyran-4-amine (38); tert-butyl 3-(methyl((1-(3-nitrophenyl)-1H-tetrazol-5-yl)methyl)amino)pyrrolidine-1-carboxylate (39): N-methyl-N-((1-(3-nitrophenyl)-1H-tetrazol-5-yl)methyl)tetrahydrofuran-3-amine (40); N-methyl-N-((1-(3-nitrophenyl)-1H-tetrazol-5-yl)methyl)aniline (41); N,2-dimethyl-N-((1-(3-nitrophenyl)-1H-tetrazol-5-yl)methyl)propan-1-amine (42); 2-(cyclohexyl((1-(3-nitrophenyl)-1H-tetrazol-5-yl)methyl)amino)ethan-1-ol (43); N-methyl-N-((1-(3-nitrophenyl)-1H-tetrazol-5-yl)methyl)cyclopropanamine (44); N-((1-(3-bromophenyl)-1H-tetrazol-5-yl)methyl)-N-methylcyclohexanamine (45); N-((1-(6-fluoropyridin-3-yl)-1H-tetrazol-5-yl)methyl)-N-methylcyclohexanamine (46): 5-(5-((cyclohexyl(methyl)amino)methyl)-1H-tetrazol-1-yl)picolinonitrile (47): 5-(5-((cyclohexyl(methyl)amino)methyl)-1H-tetrazol-1-yl)-1-methyl-2-oxo-1,2-dihydropyridine-3-carbonitrile (48): N-((1-(4-chloro-3-(trifluoromethyl)phenyl)-1H-tetrazol-5-yl)methyl)-N-methylcyclohexanamine (49); 2-chloro-5-(5-((2,5-dimethylpyrrolidin-1-yl)methyl)-1H-tetrazol-1-yl)benzonitrile (50); ethyl 4-(((1-(4-chloro-3-cyanophenyl)-1H-tetrazol-5-yl)methyl)(cyclopropyl)amino) cyclohexane-1-carboxylate (51); 2-chloro-5-(5-((cyclohexyl(2-hydroxy-2-phenylethyl) amino)methyl)-1H-tetrazol-1-yl)benzonitrile (52); 3-chloro-6-(5-((cyclohexyl(propyl) amino)methyl)-1H-tetrazol-1-yl)picolinonitrile (53): N-(3-(((1-(5-chloro-6-cyanopyridin-2-yl)-1H-tetrazol-5-yl)methyl)(cyclohexyl) amino)propyl)benzamide (54); 3-chloro-6-(5-((cyclohexyl(isopropyl)amino)methyl)-1H-tetrazol-1-yl)picolinonitrile (55); ethyl 4-(((1-(5-chloro-6-cyanopyridin-2-yl)-1H-tetrazol-5-yl)methyl)(cyclopropyl)amino) cyclohexane-1-carboxylate (56); 3-chloro-6-(5-((cyclohexyl(2-hydroxy-2-phenylethyl) amino)methyl)-1H-tetrazol-1-yl) picolinonitrile (57); 6-(5-((benzyl((1R,2R)-2-hydroxycyclohexyl)amino)methyl)-1H-tetrazol-1-yl)-3-chloropicolinonitrile (58); 3-chloro-6-(5-((cyclopropyl((1s,4s)-4-hydroxy-4-methylcyclohexyl)amino)methyl)-1H-tetrazol-1-yl)picolinonitrile (59); 3-chloro-6-(5-((isopropyl(methyl)amino)methyl)-1H-tetrazol-1-yl)picolinonitrile (60); 3-chloro-6-(5-((isobutyl(methyl)amino)methyl)-1H-tetrazol-1-yl)picolinonitrile (61); 3-chloro-6-(5-((cyclohexyl(2-hydroxyethyl)amino) methyl)-1H-tetrazol-1-v) picolinonitrile (62); or 6-(5-((butyl(cyclohexyl)amino)methyl)-1H-tetrazol-1-yl)-3-chloropicolinonitrile (63).

The present invention may be embodied in other specific forms without departing from the spirit or essential attributes thereof. This invention encompasses all combinations of the aspects and/or embodiments of the invention noted herein. It is understood that any and all embodiments of the present invention may be taken in conjunction with any other embodiment or embodiments to describe additional embodiments. It is also to be understood that each individual element of the embodiments is meant to be combined with any and all other elements from any embodiment to describe an additional embodiment.

### Definitions

The features and advantages of the invention may be more readily understood by those of ordinary skill in the art upon reading the following detailed description. It is to be appreciated that certain features of the invention that are, for clarity reasons, described above and below in the context of separate embodiments, may also be combined to form a single embodiment. Conversely, various features of the invention that are, for brevity reasons, described in the context of a single embodiment, may also be combined so as to form sub-combinations thereof. Embodiments identified herein as exemplary or preferred are intended to be illustrative and not limiting.

Unless specifically stated otherwise herein, references made in the singular may also include the plural. For example, “a” and “an” may refer to either one, or one or more.

As used herein, the phrase “compounds and/or salts thereof” refers to at least one compound, at least one salt of the compounds, or a combination thereof. For example, compounds of Formula (I) and/or salts thereof includes a compound of Formula (I): two compounds of Formula (I); a salt of a compound of Formula (I); a compound of Formula (I) and one or more salts of the compound of Formula (I); and two or more salts of a compound of Formula (I).

Unless otherwise indicated, any atom with unsatisfied valences is assumed to have hydrogen atoms sufficient to satisfy the valences.

The definitions set forth herein take precedence over definitions set forth in any patent, patent application, and/or patent application publication incorporated herein by reference.

Listed below are definitions of various terms used to describe the present invention. These definitions apply to the terms as they are used throughout the specification (unless they are otherwise limited in specific instances) either individually or as part of a larger group.

Throughout the specification, groups and substituents thereof may be chosen by one skilled in the field to provide stable moieties and compounds.

In accordance with a convention used in the art.

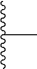

is used in structural formulas herein to depict the bond that is the point of attachment of the moiety or substituent to the core or backbone structure.

The terms “halo” and “halogen,” as used herein, refer to F, Cl, Br, and I.

The term “cyano” refers to the group —CN.

The term “amino” refers to the group —NH₂.

The term “oxo” refers to the group ═O.

The term “alkyl” as used herein, refers to both branched and straight-chain saturated aliphatic hydrocarbon groups containing, for example, from 1 to 12 carbon atoms, from 1 to 6 carbon atoms, and from 1 to 4 carbon atoms. Examples of alkyl groups include, but are not limited to, methyl (Me), ethyl (Et), propyl (e.g., n-propyl and i-propyl), butyl (e.g., n-butyl, i-butyl, sec-butyl, and t-butyl), and pentyl (e.g., n-pentyl, isopentyl, neopentyl), n-hexyl, 2-methylpentyl, 2-ethylbutyl, 3-methylpentyl, and 4-methylpentyl. When numbers appear in a subscript after the symbol “C”, the subscript defines with more specificity the number of carbon atoms that a particular group may contain. For example, “C₁₋₄ alkyl” denotes straight and branched chain alkyl groups with one to four carbon atoms.

The term “fluoroalkyl” as used herein is intended to include both branched and straight-chain saturated aliphatic hydrocarbon groups substituted with one or more fluorine atoms. For example, “C₁₋₄ fluoroalkyl” is intended to include C₁, C₂, C₃, and C₄ alkyl groups substituted with one or more fluorine atoms. Representative examples of fluoroalkyl groups include, but are not limited to, —CF₃ and —CH₂CF₃.

The term “hydroxyalkyl” includes both branched and straight-chain saturated alkyl groups substituted with one or more hydroxyl groups. For example. “hydroxyalkyl” includes —CH₂OH, —CH₂CH₂OH, and C₁₋₄ hydroxyalkyl.

The term “cycloalkyl,” as used herein, refers to a group derived from a non-aromatic monocyclic or polycyclic hydrocarbon molecule by removal of one hydrogen atom from a saturated ring carbon atom. Representative examples of cycloalkyl groups include, but are not limited to, cyclopropyl, cyclopentyl, and cyclohexyl. When numbers appear in a subscript after the symbol “C”, the subscript defines with more specificity the number of carbon atoms that a particular cycloalkyl group may contain. For example, “C₃₋₆ cycloalkyl” denotes cycloalkyl groups with three to six carbon atoms.

The term “alkoxy,” as used herein, refers to an alkyl group attached to the parent molecular moiety through an oxygen atom, for example, methoxy group (—OCH₃). For example, “C₁₋₃ alkoxy” denotes alkoxy groups with one to three carbon atoms.

The terms “fluoroalkoxy” and “—O(fluoroalkyl)” represent a fluoroalkyl group as defined above attached through an oxygen linkage (—O—). For example, “C₁₋₄ fluoroalkoxy” is intended to include C₁, C₂, C₃, and C₄ fluoroalkoxy groups.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The compounds of Formula (I) can form salts which are also within the scope of this invention. Unless otherwise indicated, reference to an inventive compound is understood to include reference to one or more salts thereof. The term “salt(s)” denotes acidic salts formed with inorganic and/or organic acids. In addition, the term “salt(s) may include zwitterions (inner salts), e.g., when a compound of Formula (I) contains both a basic moiety, such as an amine or a pyridine or imidazole ring, and an acidic moiety, such as a carboxylic acid. Pharmaceutically acceptable (i.e., non-toxic, physiologically acceptable) salts are preferred, such as, for example, acceptable metal and amine salts in which the cation does not contribute significantly to the toxicity or biological activity of the salt. However, other salts may be useful, e.g., in isolation or purification steps which may be employed during preparation, and thus, are contemplated within the scope of the invention. Salts of the compounds of the formula (I) may be formed, for example, by reacting a compound of the Formula (I) with an amount of acid, such as an equivalent amount, in a medium such as one in which the salt precipitates or in an aqueous medium followed by lyophilization.

Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides (formed with hydrochloric acid), hydrobromides (formed with hydrogen bromide), hydroiodides, maleates (formed with maleic acid), 2-hydroxyethanesulfonates, lactates, methanesulfonates (formed with methanesulfonic acid), 2-naphthalenesulfonates, nicotinates, nitrates, oxalates, pectinates, persulfates, 3-phenylpropionates, phosphates, picrates, pivalates, propionates, salicylates, succinates, sulfates (such as those formed with sulfuric acid), sulfonates (such as those mentioned herein), tartrates, thiocyanates, toluenesulfonates such as tosylates, undecanoates, and the like.

The compounds of Formula (I) can be provided as amorphous solids or crystalline solids. Lyophilization can be employed to provide the compounds of Formula (I) as a solid.

It should further be understood that solvates (e.g., hydrates) of the Compounds of Formula (I) are also within the scope of the present invention. The term “solvate” means a physical association of a compound of Formula (I) with one or more solvent molecules, whether organic or inorganic. This physical association includes hydrogen bonding. In certain instances the solvate will be capable of isolation, for example when one or more solvent molecules are incorporated in the crystal lattice of the crystalline solid. “Solvate” encompasses both solution-phase and isolable solvates. Exemplary solvates include hydrates, ethanolates, methanolates, isopropanolates, acetonitrile solvates, and ethyl acetate solvates. Methods of solvation are known in the art.

Various forms of prodrugs are known in the art and are described in Rautio, J. et al., Nature Review Drug Discovery. 17, 559-587 (2018).

In addition, compounds of Formula (I), subsequent to their preparation, can be isolated and purified to obtain a composition containing an amount by weight equal to or greater than 99% of a compound of Formula (I) (“substantially pure”), which is then used or formulated as described herein. Such “substantially pure” compounds of Formula (I) are also contemplated herein as part of the present invention.

“Stable compound” and “stable structure” are meant to indicate a compound that is sufficiently robust to survive isolation to a useful degree of purity from a reaction mixture, and formulation into an efficacious therapeutic agent. The present invention is intended to embody stable compounds.

“Therapeutically effective amount” is intended to include an amount of a compound of the present invention alone or an amount of the combination of compounds claimed or an amount of a compound of the present invention in combination with other active ingredients effective to act as an inhibitor of DGKα and/or DGKζ, or effective to treat or prevent viral infections and proliferative disorders, such as cancer.

As used herein, “treating” or “treatment” cover the treatment of a disease-state in a mammal, particularly in a human, and include: (a) preventing the disease-state from occurring in a mammal, in particular, when such mammal is predisposed to the disease-state but has not yet been diagnosed as having it; (b) inhibiting the disease-state, i.e., arresting its development; and/or (c) relieving the disease-state, i.e., causing regression of the disease state.

The compounds of the present invention are intended to include all isotopes of atoms occurring in the present compounds. Isotopes include those atoms having the same atomic number but different mass numbers. By way of general example and without limitation, isotopes of hydrogen include deuterium (D) and tritium (T). Isotopes of carbon include ¹³C and ¹⁴C. Isotopically-labeled compounds of the invention can generally be prepared by conventional techniques known to those skilled in the art or by processes analogous to those described herein, using an appropriate isotopically-labeled reagent in place of the non-labeled reagent otherwise employed.

Compounds in accordance with Formula (I) and/or pharmaceutically acceptable salts thereof can be administered by any means suitable for the condition to be treated, which can depend on the need for site-specific treatment or quantity of Formula (I) compound to be delivered.

Also embraced within this invention is a class of pharmaceutical compositions comprising a compound of Formula (I) and/or pharmaceutically acceptable salts thereof; and one or more non-toxic, pharmaceutically-acceptable carriers and/or diluents and/or adjuvants (collectively referred to herein as “carrier” materials) and, if desired, other active ingredients. The compounds of Formula (I) may be administered by any suitable route, preferably in the form of a pharmaceutical composition adapted to such a route, and in a dose effective for the treatment intended. The compounds and compositions of the present invention may, for example, be administered orally, mucosally, or parentally including intravascularly, intravenously, intraperitoneally, subcutaneously, intramuscularly, and intrasternally in dosage unit formulations containing conventional pharmaceutically acceptable carriers, adjuvants, and vehicles. For example, the pharmaceutical carrier may contain a mixture of mannitol or lactose and microcrystalline cellulose. The mixture may contain additional components such as a lubricating agent, e.g. magnesium stearate and a disintegrating agent such as crospovidone. The carrier mixture may be filled into a gelatin capsule or compressed as a tablet. The pharmaceutical composition may be administered as an oral dosage form or an infusion, for example.

For oral administration, the pharmaceutical composition may be in the form of, for example, a tablet, capsule, liquid capsule, suspension, or liquid. The pharmaceutical composition is preferably made in the form of a dosage unit containing a particular amount of the active ingredient. For example, the pharmaceutical composition may be provided as a tablet or capsule comprising an amount of active ingredient in the range of from about 0.1 to 1000 mg, preferably from about 0.25 to 250 mg, and more preferably from about 0.5 to 100 mg. A suitable daily dose for a human or other mammal may vary widely depending on the condition of the patient and other factors, but, can be determined using routine methods.

Any pharmaceutical composition contemplated herein can, for example, be delivered orally via any acceptable and suitable oral preparations. Exemplary oral preparations, include, but are not limited to, for example, tablets, troches, lozenges, aqueous and oily suspensions, dispersible powders or granules, emulsions, hard and soft capsules, liquid capsules, syrups, and elixirs. Pharmaceutical compositions intended for oral administration can be prepared according to any methods known in the art for manufacturing pharmaceutical compositions intended for oral administration. In order to provide pharmaceutically palatable preparations, a pharmaceutical composition in accordance with the invention can contain at least one agent selected from sweetening agents, flavoring agents, coloring agents, demulcents, antioxidants, and preserving agents.

A tablet can, for example, be prepared by admixing at least one compound of Formula (I) and/or at least one pharmaceutically acceptable salt thereof with at least one non-toxic pharmaceutically acceptable excipient suitable for the manufacture of tablets. Exemplary excipients include, but are not limited to, for example, inert diluents, such as, for example, calcium carbonate, sodium carbonate, lactose, calcium phosphate, and sodium phosphate; granulating and disintegrating agents, such as, for example, microcrystalline cellulose, sodium crosscarmellose, corn starch, and alginic acid; binding agents, such as, for example, starch, gelatin, polyvinyl-pyrrolidone, and acacia; and lubricating agents, such as, for example, magnesium stearate, stearic acid, and talc. Additionally, a tablet can either be uncoated, or coated by known techniques to either mask the bad taste of an unpleasant tasting drug, or delay disintegration and absorption of the active ingredient in the gastrointestinal tract thereby sustaining the effects of the active ingredient for a longer period. Exemplary water soluble taste masking materials, include, but are not limited to, hydroxypropyl-methylcellulose and hydroxypropyl-cellulose. Exemplary time delay materials, include, but are not limited to, ethyl cellulose and cellulose acetate butyrate.

Hard gelatin capsules can, for example, be prepared by mixing at least one compound of Formula (I) and/or at least one salt thereof with at least one inert solid diluent, such as, for example, calcium carbonate; calcium phosphate; and kaolin.

Soft gelatin capsules can, for example, be prepared by mixing at least one compound of Formula (I) and/or at least one pharmaceutically acceptable salt thereof with at least one water soluble carrier, such as, for example, polyethylene glycol; and at least one oil medium, such as, for example, peanut oil, liquid paraffin, and olive oil.

An aqueous suspension can be prepared, for example, by admixing at least one compound of Formula (I) and/or at least one pharmaceutically acceptable salt thereof with at least one excipient suitable for the manufacture of an aqueous suspension. Exemplary excipients suitable for the manufacture of an aqueous suspension, include, but are not limited to, for example, suspending agents, such as, for example, sodium carboxymethylcellulose, methylcellulose, hydroxypropylmethyl-cellulose, sodium alginate, alginic acid, polyvinyl-pyrrolidone, gum tragacanth, and gum acacia, dispersing or wetting agents, such as, for example, a naturally-occurring phosphatide, e.g., lecithin; condensation products of alkylene oxide with fatty acids, such as, for example, polyoxyethylene stearate; condensation products of ethylene oxide with long chain aliphatic alcohols, such as, for example heptadecaethylene-oxycetanol; condensation products of ethylene oxide with partial esters derived from fatty acids and hexitol, such as, for example, polyoxyethylene sorbitol monooleate; and condensation products of ethylene oxide with partial esters derived from fatty acids and hexitol anhydrides, such as, for example, polyethylene sorbitan monooleate. An aqueous suspension can also contain at least one preservative, such as, for example, ethyl and n-propyl p-hydroxybenzoate; at least one coloring agent; at least one flavoring agent; and/or at least one sweetening agent, including but not limited to, for example, sucrose, saccharin, and aspartame.

Oily suspensions can, for example, be prepared by suspending at least one compound of Formula (I) and/or at least one pharmaceutically acceptable salt thereof in either a vegetable oil, such as, for example, arachis oil; olive oil; sesame oil; and coconut oil, or in mineral oil, such as, for example, liquid paraffin. An oily suspension can also contain at least one thickening agent, such as, for example, beeswax; hard paraffin; and cetyl alcohol. In order to provide a palatable oily suspension, at least one of the sweetening agents already described hereinabove, and/or at least one flavoring agent can be added to the oily suspension. An oily suspension can further contain at least one preservative, including, but not limited to, for example, an anti-oxidant, such as, for example, butylated hydroxyanisol, and alpha-tocopherol.

Dispersible powders and granules can, for example, be prepared by admixing at least one compound of Formula (I) and/or at least one pharmaceutically acceptable salt thereof with at least one dispersing and/or wetting agent; at least one suspending agent; and/or at least one preservative. Suitable dispersing agents, wetting agents, and suspending agents are as already described above. Exemplary preservatives include, but are not limited to, for example, anti-oxidants, e.g., ascorbic acid. In addition, dispersible powders and granules can also contain at least one excipient, including, but not limited to, for example, sweetening agents, flavoring agents; and coloring agents.

An emulsion of at least one compound of Formula (I) and/or at least one pharmaceutically acceptable salt thereof can, for example, be prepared as an oil-in-water emulsion. The oily phase of the emulsions comprising compounds of Formula (I) may be constituted from known ingredients in a known manner. The oil phase can be provided by, but is not limited to, for example, a vegetable oil, such as, for example, olive oil and arachis oil; a mineral oil, such as, for example, liquid paraffin; and mixtures thereof. While the phase may comprise merely an emulsifier, it may comprise a mixture of at least one emulsifier with a fat or an oil or with both a fat and an oil. Suitable emulsifying agents include, but are not limited to, for example, naturally-occurring phosphatides, e.g., soy bean lecithin; esters or partial esters derived from fatty acids and hexitol anhydrides, such as, for example, sorbitan monooleate; and condensation products of partial esters with ethylene oxide, such as, for example, polyoxyethylene sorbitan monooleate. Preferably, a hydrophilic emulsifier is included together with a lipophilic emulsifier which acts as a stabilizer. It is also preferred to include both an oil and a fat. Together, the emulsifier(s) with or without stabilizer(s) make-up the so-called emulsifying wax, and the wax together with the oil and fat make up the so-called emulsifying ointment base which forms the oily dispersed phase of the cream formulations. An emulsion can also contain a sweetening agent, a flavoring agent, a preservative, and/or an antioxidant. Emulsifiers and emulsion stabilizers suitable for use in the formulation of the present invention include Tween 60, Span 80, cetostearyl alcohol, myristyl alcohol, glyceryl monostearate, sodium lauryl sulfate, glyceryl distearate alone or with a wax, or other materials well known in the art.

The compounds of Formula (I) and/or at least one pharmaceutically acceptable salt thereof can, for example, also be delivered intravenously, subcutaneously, and/or intramuscularly via any pharmaceutically acceptable and suitable injectable form. Exemplary injectable forms include, but are not limited to, for example, sterile aqueous solutions comprising acceptable vehicles and solvents, such as, for example, water, Ringer's solution, and isotonic sodium chloride solution; sterile oil-in-water microemulsions; and aqueous or oleaginous suspensions.

Formulations for parenteral administration may be in the form of aqueous or non-aqueous isotonic sterile injection solutions or suspensions. These solutions and suspensions may be prepared from sterile powders or granules using one or more of the carriers or diluents mentioned for use in the formulations for oral administration or by using other suitable dispersing or wetting agents and suspending agents. The compounds may be dissolved in water, polyethylene glycol, propylene glycol, ethanol, corn oil, cottonseed oil, peanut oil, sesame oil, benzyl alcohol, sodium chloride, tragacanth gum, and/or various buffers. Other adjuvants and modes of administration are well and widely known in the pharmaceutical art. The active ingredient may also be administered by injection as a composition with suitable carriers including saline, dextrose, or water, or with cyclodextrin (i.e. Captisol), cosolvent solubilization (i.e. propylene glycol) or micellar solubilization (i.e. Tween 80).

The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed, including synthetic mono- or diglycerides. In addition, fatty acids such as oleic acid find use in the preparation of injectables.

A sterile injectable oil-in-water microemulsion can, for example, be prepared by 1) dissolving at least one compound of Formula (I) in an oily phase, such as, for example, a mixture of soybean oil and lecithin: 2) combining the Formula (I) containing oil phase with a water and glycerol mixture; and 3) processing the combination to form a microemulsion.

A sterile aqueous or oleaginous suspension can be prepared in accordance with methods already known in the art. For example, a sterile aqueous solution or suspension can be prepared with a non-toxic parenterally-acceptable diluent or solvent, such as, for example, 1,3-butane diol; and a sterile oleaginous suspension can be prepared with a sterile non-toxic acceptable solvent or suspending medium, such as, for example, sterile fixed oils, e.g., synthetic mono- or diglycerides; and fatty acids, such as, for example, oleic acid.

Pharmaceutically acceptable carriers, adjuvants, and vehicles that may be used in the pharmaceutical compositions of this invention include, but are not limited to, ion exchangers, alumina, aluminum stearate, lecithin, self-emulsifying drug delivery systems (SEDDS) such as d-alpha-tocopherol polyethyleneglycol 1000 succinate, surfactants used in pharmaceutical dosage forms such as Tweens, polyethoxylated castor oil such as CREMOPHOR surfactant (BASF), or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as alpha-, beta-, and gamma-cyclodextrin, or chemically modified derivatives such as hydroxyalkylcyclodextrins, including 2- and 3-hydroxypropyl-cyclodextrins, or other solubilized derivatives may also be advantageously used to enhance delivery of compounds of the formulae described herein.

The pharmaceutically active compounds of this invention can be processed in accordance with conventional methods of pharmacy to produce medicinal agents for administration to patients, including humans and other mammals. The pharmaceutical compositions may be subjected to conventional pharmaceutical operations such as sterilization and/or may contain conventional adjuvants, such as preservatives, stabilizers, wetting agents, emulsifiers, buffers etc. Tablets and pills can additionally be prepared with enteric coatings. Such compositions may also comprise adjuvants, such as wetting, sweetening, flavoring, and perfuming agents.

The amounts of compounds that are administered and the dosage regimen for treating a disease condition with the compounds and/or compositions of this invention depends on a variety of factors, including the age, weight, sex, the medical condition of the subject, the type of disease, the severity of the disease, the route and frequency of administration, and the particular compound employed. Thus, the dosage regimen may vary widely, but can be determined routinely using standard methods. A daily dose of about 0.001 to 100 mg/kg body weight, preferably between about 0.0025 and about 50 mg/kg body weight and most preferably between about 0.005 to 10 mg/kg body weight, may be appropriate. The daily dose can be administered in one to four doses per day. Other dosing schedules include one dose per week and one dose per two day cycle.

For therapeutic purposes, the active compounds of this invention are ordinarily combined with one or more adjuvants appropriate to the indicated route of administration. If administered orally, the compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation as may be provided in a dispersion of active compound in hydroxypropylmethyl cellulose.

Pharmaceutical compositions of this invention comprise at least one compound of Formula (I) and/or at least one pharmaceutically acceptable salt thereof, and optionally an additional agent selected from any pharmaceutically acceptable carrier, adjuvant, and vehicle. Alternate compositions of this invention comprise a compound of the Formula (I) described herein, or a prodrug thereof, and a pharmaceutically acceptable carrier, adjuvant, or vehicle.

### UTILITY

The compounds of Formula (I) are useful for the treatment of cancer.

In another embodiment, the present invention provides a combined preparation of a compound of Formula (I), and/or a pharmaceutically acceptable salt thereof, a stereoisomer thereof or a tautomer thereof, and additional therapeutic agent(s) for simultaneous, separate or sequential use in the treatment and/or prophylaxis of multiple diseases or disorders associated with DGK target inhibition in T cells.

In another aspect, the invention provides a method of treating a patient suffering from or susceptible to a medical condition that is associated with DGK target inhibition in T cells. A number of medical conditions can be treated. The method comprises administering to the patient a therapeutically effective amount of a composition comprising a compound of Formula (I) and/or a pharmaceutically acceptable salt thereof, a stereoisomer thereof or a tautomer thereof. For example, the compounds described herein may be used to treat or prevent viral infections and proliferative diseases such as cancer.

The compounds for Formula (I) and pharmaceutical compositions comprising at least one compound of Formula (I) are useful in treating or preventing any disease or conditions that are associated with DGK target inhibition in T cells. These include viral and other infections (e.g., skin infections, GI infection, urinary tract infections, genito-urinary infections, systemic infections), and proliferative diseases (e.g., cancer). The compounds of Formula (I) and pharmaceutical compositions comprising in at least one compound of Formula (I) may be administered to animals, preferably mammals (e.g., domesticated animals, cats, dogs, mice, rats), and more preferably humans. Any method of administration may be used to deliver the compound or pharmaceutical composition to the patient. In certain embodiments, the compound of Formula (I) or pharmaceutical composition comprising at least compound of Formula (I) is administered orally. In other embodiments, the Formula (I) or pharmaceutical composition comprising at least compound of Formula (I) is administered parenterally.

The compounds of Formula (I) can inhibit activity of the diacylglycerol kinase alpha and zeta (DGKα/C). For example, the compounds of Formula (I) can be used to inhibit activity of DGKα and DGKζ in a cell or in an individual in need of modulation of DGKα and DGKζ by administering an inhibiting amount of a compound of Formula (I) or a salt thereof.

The present invention further provides methods of treating diseases associated with activity or expression, including abnormal activity and/or overexpression, of DGKα and DGKζ in an individual (e.g., patient) by administering to the individual in need of such treatment a therapeutically effective amount or dose of a compound of Formula (I) or a pharmaceutical composition thereof. Example diseases can include any disease, disorder or condition that is directly or indirectly linked to expression or activity of DGKα and DGKζ enzyme, such as over expression or abnormal activity. A DGKα and DGKζ-associated disease can also include any disease, disorder or condition that can be prevented, ameliorated, or cured by modulating DGKα and DGKζ enzyme activity. Examples of DGKα and DGKζ associated diseases include cancer and viral infections such as HIV infection, hepatitis B, and hepatitis C.

In one aspect, the compound(s) of Formula (I) are sequentially administered prior to administration of the immuno-oncology agent. In another aspect, compound(s) of Formula (I) are administered concurrently with the immuno-oncology agent. In yet another aspect, compound(s) of Formula (I) are sequentially administered after administration of the immuno-oncology agent.

In another aspect, compounds of Formula (I) may be co-formulated with an immuno-oncology agent.

Immuno-oncology agents include, for example, a small molecule drug, antibody, or other biologic or small molecule. Examples of biologic immuno-oncology agents include, but are not limited to, cancer vaccines, antibodies, and cytokines. In one aspect, the antibody is a monoclonal antibody. In another aspect, the monoclonal antibody is humanized or human.

In one aspect, the immuno-oncology agent is (i) an agonist of a stimulatory (including a co-stimulatory) receptor or (ii) an antagonist of an inhibitory (including a co-inhibitory) signal on T cells, both of which result in amplifying antigen-specific T cell responses (often referred to as immune checkpoint regulators).

Certain of the stimulatory and inhibitory molecules are members of the immunoglobulin super family (IgSF). One important family of membrane-bound ligands that bind to co-stimulatory or co-inhibitory receptors is the B7 family, which includes B7-1, B7-2, B7-H1 (PD-L1), B7-DC (PD-L2), B7-H2 (ICOS-L), B7-H3, B7-H4, B7-H5 (VISTA), and B7-H6. Another family of membrane bound ligands that bind to co-stimulatory or co-inhibitory receptors is the TNF family of molecules that bind to cognate TNF receptor family members, which includes CD40 and CD40L, OX-40, OX-40L, CD70, CD27L, CD30, CD30L, 4-1BBL, CD137 (4-1BB), TRAIIJApo2-L, TRAILR 1/DR4, TRAILR2/DR5, TRAILR3, TRAILR4, OPG, RANK, RANKL, TWEAKR/Fn14, TWEAK, BAFFR, EDAR, XEDAR, TACI, APRIL, BCMA, LTβR, LIGHT, DcR3, HVEM, VEGIUTLIA, TRAMP/DR3, EDAR, EDA1, XEDAR, EDA2, TNFRL, Lymphotoxin α/TNFβ, TNFR2, TNFα, LTPR, Lymphotoxin α 1β2, FAS, FASL, RELT, DR6, TROY, NGFR.

In one aspect, T cell responses can be stimulated by a combination of a compound of Formula (I) and one or more of (i) an antagonist of a protein that inhibits T cell activation (e.g., immune checkpoint inhibitors) such as CTLA-4, PD-1, PD-L1, PD-L2, LAG-3, TIM-3, Galectin 9. CEACAM-1, BTLA. CD69, Galectin-1, TIGIT, CD113, GPR56, VISTA, 2B4, CD48, GARP, PDIH, LAIR1, TIM-1, and TIM-4, and (ii) an agonist of a protein that stimulates T cell activation such as B7-1, B7-2, CD28, 4-1BB (CD137), 4-1BBL, ICOS, ICOS-L, OX40, OX40L, GITR. GITRL, CD70, CD27, CD40, DR3 and CD28H.

Other agents that can be combined with compounds of Formula (I) for the treatment of cancer include antagonists of inhibitory receptors on NK cells or agonists of activating receptors on NK cells. For example, compounds of Formula (I) can be combined with antagonists of KIR, such as lirilumab.

Yet other agents for combination therapies include agents that inhibit or deplete macrophages or monocytes, including but not limited to CSF-1R antagonists such as CSF-1R antagonist antibodies including RG7155 (WO11/70024, WO11/107553, WO11/131407, WO13/87699, WO13/119716, WO13/132044) or FPA-008 (WO11/140249: WO13169264; WO14/036357).

In another aspect, compounds of Formula (I) can be used with one or more of agonistic agents that ligate positive costimulatory receptors, blocking agents that attenuate signaling through inhibitory receptors, antagonists, and one or more agents that increase systemically the frequency of anti-tumor T cells, agents that overcome distinct immune suppressive pathways within the tumor microenvironment (e.g., block inhibitory receptor engagement (e.g., PD-L1/PD-1 interactions), deplete or inhibit Tregs (e.g., using an anti-CD25 monoclonal antibody (e.g., daclizumab) or by ex vivo anti-CD25 bead depletion), inhibit metabolic enzymes such as IDO, or reverse/prevent T cell anergy or exhaustion) and agents that trigger innate immune activation and/or inflammation at tumor sites.

In one aspect, the immuno-oncology agent is a CTLA-4 antagonist, such as an antagonistic CTLA-4 antibody. Suitable CTLA-4 antibodies include, for example, YERVOY (ipilimumab) or tremelimumab.

In another aspect, the immuno-oncology agent is a PD-1 antagonist, such as an antagonistic PD-1 antibody. Suitable PD-1 antibodies include, for example OPDIVO (nivolumab), KEYTRUDA (pembrolizumab), or MEDI-0680 (AMP-514; WO2012/145493). The immuno-oncology agent may also include pidilizumab (CT-011), though its specificity for PD-1 binding has been questioned. Another approach to target the PD-1 receptor is the recombinant protein composed of the extracellular domain of PD-L2 (B7-DC) fused to the Fc portion of IgG1, called AMP-224

In another aspect, the immuno-oncology agent is a PD-L1 antagonist, such as an antagonistic PD-L1 antibody. Suitable PD-L1 antibodies include, for example, MPDL3280A (RG7446; WO2010/077634), durvalumab (MED14736), BMS-936559 (WO2007/005874), and MSBOO10718C (WO2013/79174).

In another aspect, the immuno-oncology agent is a LAG-3 antagonist, such as an antagonistic LAG-3 antibody. Suitable LAG3 antibodies include, for example, BMS-986016 (WO10/19570, WO14/08218), or IMP-731 or IMP-321 (WO08/132601, WO09/44273).

In another aspect, the immuno-oncology agent is a CD137 (4-1BB) agonist, such as an agonistic CD137 antibody. Suitable CD137 antibodies include, for example, urelumab and PF-05082566 (WO12/32433).

In another aspect, the immuno-oncology agent is a GITR agonist, such as an agonistic GITR antibody. Suitable GITR antibodies include, for example, BMS-986153, BMS-986156, TRX-518 (WO06/105021, WO09/009116) and MK-4166 (WO11/028683).

In another aspect, the immuno-oncology agent is an IDO antagonist. Suitable IDO antagonists include, for example, INCB-024360 (WO2006/122150, WO07/75598, WO08/36653, WO08/36642), indoximod, BMS-986205, or NLG-919 (WO09/73620, WO09/1156652, WO11/56652, WO12/142237).

In another aspect, the immuno-oncology agent is an OX40 agonist, such as an agonistic OX40 antibody. Suitable OX40 antibodies include, for example, MEDI-6383 or MEDI-6469.

In another aspect, the immuno-oncology agent is an OX40L antagonist, such as an antagonistic OX40 antibody. Suitable OX40L antagonists include, for example, RG-7888 (WO06/029879).

In another aspect, the immuno-oncology agent is a CD40 agonist, such as an agonistic CD40 antibody. In yet another embodiment, the immuno-oncology agent is a CD40 antagonist, such as an antagonistic CD40 antibody. Suitable CD40 antibodies include, for example, lucatumumab or dacetuzumab.

In another aspect, the immuno-oncology agent is a CD27 agonist, such as an agonistic CD27 antibody. Suitable CD27 antibodies include, for example, varlilumab.

In another aspect, the immuno-oncology agent is MGA271 (to B7H3) (WO1/109400).

The combination therapy is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single dosage form having a fixed ratio of each therapeutic agent or in multiple, single dosage forms for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. Combination therapy also can embrace the administration of the therapeutic agents as described above in further combination with other biologically active ingredients and non-drug therapies (e.g., surgery or radiation treatment.) Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “contacting” refers to the bringing together of indicated moieties in an in vitro system or an in vivo system. For example, “contacting” the DGKα and DGKζ enzyme with a compound of Formula (I) includes the administration of a compound of the present invention to an individual or patient, such as a human, having DGKα and DGKζ, as well as, for example, introducing a compound of Formula (I) into a sample containing a cellular or purified preparation containing DGKα and DGKζ enzyme.

The term “DGKα and DGKζ inhibitor” refers to an agent capable of inhibiting the activity of diacylglycerol kinase alpha and/or diacylglycerol kinase zeta (DGKα and DGKζ) in T cells resulting in T cell stimulation. The DGKα and DGKζ inhibitor may be a reversible or irreversible DGKα and DGKζ inhibitor. “A reversible DGKα and DGKζ inhibitor” is a compound that reversibly inhibits DGKα and DGKζ enzyme activity either at the catalytic site or at a non-catalytic site and “an irreversible DGKα and DGKζ inhibitor” is a compound that irreversibly destroys DGKα and DGKζ enzyme activity by forming a covalent bond with the enzyme.

Types of cancers that may be treated with the compound of Formula (I) include, but are not limited to, brain cancers, skin cancers, bladder cancers, ovarian cancers, breast cancers, gastric cancers, pancreatic cancers, prostate cancers, colon cancers, blood cancers, lung cancers and bone cancers. Examples of such cancer types include neuroblastoma, intestine carcinoma such as rectum carcinoma, colon carcinoma, familiar adenomatous polyposis carcinoma and hereditary non-polyposis colorectal cancer, esophageal carcinoma, labial carcinoma, larynx carcinoma, hypopharynx carcinoma, tongue carcinoma, salivary gland carcinoma, gastric carcinoma, adenocarcinoma, medullary thyroid carcinoma, papillary thyroid carcinoma, renal carcinoma, kidney parenchymal carcinoma, ovarian carcinoma, cervix carcinoma, uterine corpus carcinoma, endometrium carcinoma, chorion carcinoma, pancreatic carcinoma, prostate carcinoma, testis carcinoma, breast carcinoma, urinary carcinoma, melanoma, brain tumors such as glioblastoma, astrocytoma, meningioma, medulloblastoma and peripheral neuroectodermal tumors, Hodgkin lymphoma, non-Hodgkin lymphoma, Burkitt lymphoma, acute lymphatic leukemia (ALL), chronic lymphatic leukemia (CLL), acute myeloid leukemia (AML), chronic mycloid leukemia (CML), adult T-cell leukemia lymphoma, diffuse large B-cell lymphoma (DLBCL), hepatocellular carcinoma, gall bladder carcinoma, bronchial carcinoma, small cell lung carcinoma, non-small cell lung carcinoma, multiple mycloma, basalioma, teratoma, retinoblastoma, choroid melanoma, seminoma, rhabdomyosarcoma, craniopharyngioma, osteosarcoma, chondrosarcoma, myosarcoma, liposarcoma, fibrosarcoma, Ewing sarcoma and plasmocytoma.

One or more additional pharmaceutical agents or treatment methods such as, for example, anti-viral agents, chemotherapeutics or other anti-cancer agents, immune enhancers, immunosuppressants, radiation, anti-tumor and anti-viral vaccines, cytokine therapy (e.g., IL2 and GM-CSF), and/or tyrosine kinase inhibitors can be optionally used in combination with the compounds of Formula (I) for treatment of DGKα and DGKζ associated diseases, disorders or conditions. The agents can be combined with the present compounds in a single dosage form, or the agents can be administered simultaneously or sequentially as separate dosage forms.

Suitable chemotherapeutic or other anti-cancer agents include, for example, alkylating agents (including, without limitation, nitrogen mustards, ethylenimine derivatives, alkyl sulfonates, nitrosoureas and triazenes) such as uracil mustard, chlormethine, cyclophosphamide (CYTOXAN®), ifosfamide, melphalan, chlorambucil, pipobroman, triethylene-melamine, triethylenethiophosphoramine, busulfan, carmustine, lomustine, streptozocin, dacarbazine, and temozolomide.

In the treatment of melanoma, suitable agents for use in combination with the compounds of Formula (I) include: dacarbazine (DTIC), optionally, along with other chemotherapy drugs such as carmustine (BCNU) and cisplatin; the “Dartmouth regimen”, which consists of DTIC, BCNU, cisplatin and tamoxifen; a combination of cisplatin, vinblastine, and DTIC, temozolomide or YERVOY™. Compounds of Formula (I) may also be combined with immunotherapy drugs, including cytokines such as interferon alpha, interleukin 2, and tumor necrosis factor (TNF) in the treatment of melanoma.

Compounds of Formula (I) may also be used in combination with vaccine therapy in the treatment of melanoma. Antimelanoma vaccines are, in some ways, similar to the anti-virus vaccines which are used to prevent diseases caused by viruses such as polio, measles, and mumps. Weakened melanoma cells or parts of melanoma cells called antigens may be injected into a patient to stimulate the body's immune system to destroy melanoma cells.

Melanomas that are confined to the arms or legs may also be treated with a combination of agents including one or more compounds of Formula (I), using a hyperthermic isolated limb perfusion technique. This treatment protocol temporarily separates the circulation of the involved limb from the rest of the body and injects high doses of chemotherapy into the artery feeding the limb, thus providing high doses to the area of the tumor without exposing internal organs to these doses that might otherwise cause severe side effects. Usually the fluid is warmed to 38.9° C. to 40° C. Melphalan is the drug most often used in this chemotherapy procedure. This can be given with another agent called tumor necrosis factor (TNF).

Suitable chemotherapeutic or other anti-cancer agents include, for example, antimetabolites (including, without limitation, folic acid antagonists, pyrimidine analogs, purine analogs and adenosine deaminase inhibitors) such as methotrexate, 5-fluorouracil, floxuridine, cytarabine, 6-mercaptopurine, 6-thioguanine, fludarabine phosphate, pentostatine, and gemcitabine.

Suitable chemotherapeutic or other anti-cancer agents further include, for example, certain natural products and their derivatives (for example, vinca alkaloids, antitumor antibiotics, enzymes, lymphokines and epipodophyllotoxins) such as vinblastine, vincristine, vindesine, bleomycin, dactinomycin, daunorubicin, doxorubicin, epirubicin, idarubicin, ara-C, paclitaxel (Taxol), mithramycin, deoxyco-formycin, mitomycin-C, L-asparaginase, interferons (especially IFN-a), etoposide, and teniposide.

Other cytotoxic agents include navelbene, CPT-11, anastrazole, letrazole, capecitabine, reloxafine, and droloxafine.

Also suitable are cytotoxic agents such as epidophyllotoxin; an antineoplastic enzyme; a topoisomerase inhibitor; procarbazine; mitoxantrone; platinum coordination complexes such as cisplatin and carboplatin; biological response modifiers; growth inhibitors; antihormonal therapeutic agents; leucovorin: tegafur; and haematopoietic growth factors.

Other anti-cancer agent(s) include antibody therapeutics such as trastuzumab (HERCEPTIN®), antibodies to costimulatory molecules such as CTLA-4, 4-1BB and PD-1, or antibodies to cytokines (IL-1O or TGF-β).

Other anti-cancer agents also include those that block immune cell migration such as antagonists to chemokine receptors, including CCR2 and CCR4.

Other anti-cancer agents also include those that augment the immune system such as adjuvants or adoptive T cell transfer.

Anti-cancer vaccines include dendritic cells, synthetic peptides, DNA vaccines and recombinant viruses.

The pharmaceutical composition of the invention may optionally include at least one signal transduction inhibitor (STI). A “signal transduction inhibitor” is an agent that selectively inhibits one or more vital steps in signaling pathways, in the normal function of cancer cells, thereby leading to apoptosis. Suitable STIs include, but are not limited to: (i) bcr/abl kinase inhibitors such as, for example, STI 571 (GLEEVEC®); (ii) epidermal growth factor (EGF) receptor inhibitors such as, for example, kinase inhibitors (IRESSA®, SSI-774) and antibodies (Imelone: C225 [Goldstein et al., Clin. Cancer Res., 1:1311-1318 (1995)], and Abgenix: ABX-EGF); (iii) her-2/neu receptor inhibitors such as farnesyl transferase inhibitors (FTI) such as, for example, L-744,832 (Kohl et al., Nat. Med., 1(8):792-797 (1995)); (iv) inhibitors of Akt family kinases or the Akt pathway, such as, for example, rapamycin (see, for example, Sekulic et al., Cancer Res., 60:3504-3513 (2000)); (v) cell cycle kinase inhibitors such as, for example, flavopiridol and UCN-O1 (see, for example. Sausville, Curr. Med. Chem. Anti-Canc. Agents, 3:47-56 (2003)), and (vi) phosphatidyl inositol kinase inhibitors such as, for example, LY294002 (see, for example, Vlahos et al., J. Biol. Chem., 269:5241-5248 (1994)). Alternatively, at least one STI and at least one compound of Formula (I) may be in separate pharmaceutical compositions. In a specific embodiment of the present invention, at least one compound of Formula (I) and at least one STI may be administered to the patient concurrently or sequentially. In other words, at least one compound of Formula (I) may be administered first, at least one STI may be administered first, or at least one compound of Formula (I) and at least one STI may be administered at the same time. Additionally, when more than one compound of Formula (I) and/or STI is used, the compounds may be administered in any order.

The present invention further provides a pharmaceutical composition for the treatment of a chronic viral infection in a patient comprising at least one compound of Formula (I), optionally, at least one chemotherapeutic drug, and, optionally, at least one antiviral agent, in a pharmaceutically acceptable carrier.

Also provided is a method for treating a chronic viral infection in a patient by administering an effective amount of the above pharmaceutical composition.

In a specific embodiment of the present invention, at least one compound of Formula (I) and at least one chemotherapeutic agent are administered to the patient concurrently or sequentially. In other words, at least one compound of Formula (I) may be administered first, at least one chemotherapeutic agent may be administered first, or at least one compound of Formula (I) and the at least one STI may be administered at the same time. Additionally, when more than one compound of Formula (I) and/or chemotherapeutic agent is used, the compounds may be administered in any order. Similarly, any antiviral agent or STI may also be administered at any point in comparison to the administration of the compound of Formula (I).

Chronic viral infections that may be treated using the present combinatorial treatment include, but are not limited to, diseases caused by: hepatitis C virus (HCV), human papilloma virus (HPV), cytomegalovirus (CMV), herpes simplex virus (HSV), Epstein-Barr virus (EBV), varicella zoster virus, coxsackie virus, human immunodeficiency virus (HIV). Notably, parasitic infections (e.g., malaria) may also be treated by the above methods wherein compounds known to treat the parasitic conditions are optionally added in place of the antiviral agents.

Suitable antiviral agents contemplated for use in combination with the compound of Formula (I) can comprise nucleoside and nucleotide reverse transcriptase inhibitors (NRTIs), non-nucleoside reverse transcriptase inhibitors (NNRTIs), protease inhibitors and other antiviral drugs.

Examples of suitable NRTIs include zidovudine (AZT); didanosine (ddl); zalcitabine (ddC); stavudine (d4T); lamivudine (3TC); abacavir (1592U89); adefovir dipivoxil [bis(POM)-PMEA]; lobucavir; BCH-10652; emitricitabine [(−)-FTC]; beta-L-FD4 (also called beta-L-D4C and named beta-L-2′,3′-dideoxy-5-fluoro-cytidene): DAPD, ((−)-beta-D-2,6-diamino-purine dioxolane); and lodenosine (FddA). Typical suitable NNRTIs include nevirapine (BI-RG-587); delaviradine (BHAP, U-90152); efavirenz (DMP-266); PNU-142721; AG-1549; MKC-442 (1-(ethoxy-methyl)-5-(I-methylethyl)-6-(phenylmethyl)-(2,4(1H,3H)-pyrimidinedione); and (+)-calanolide A (NSC-675451) and B. Typical suitable protease inhibitors include saquinavir (Ro 31-8959); ritonavir (ABT-538); indinavir (MK-639); nelfnavir (AG-1343); amprenavir (141W94); lasinavir; DMP-450; BMS-2322623; ABT-378; and AG-1549. Other antiviral agents include hydroxyurea, ribavirin, IL-2, IL-12, pentafuside and Yissum Project No. 11607.

The present invention also includes pharmaceutical kits useful, for example, in the treatment or prevention of DGKα and DGKζ-associated diseases or disorders, and other diseases referred to herein which include one or more containers containing a pharmaceutical composition comprising a therapeutically effective amount of a compound of Formula (I). Such kits can further include, if desired, one or more of various conventional pharmaceutical kit components, such as, for example, containers with one or more pharmaceutically acceptable carriers, additional containers, as will be readily apparent to those skilled in the art. Instructions, either as inserts or as labels, indicating quantities of the components to be administered, guidelines for administration, and/or guidelines for mixing the components, can also be included in the kit.

The combination therapy is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single dosage form having a fixed ratio of each therapeutic agent or in multiple, single dosage forms for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. Combination therapy also can embrace the administration of the therapeutic agents as described above in further combination with other biologically active ingredients and non-drug therapies (e.g., surgery or radiation treatment). Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

The invention also provides pharmaceutically acceptable compositions which comprise a therapeutically effective amount of one or more of the compounds of Formula (I), formulated together with one or more pharmaceutically acceptable carriers (additives) and/or diluents, and optionally, one or more additional therapeutic agents described above.

The compounds of this invention can be administered for any of the uses described herein by any suitable means, for example, orally, such as tablets, capsules (each of which includes sustained release or timed release formulations), pills, powders, granules, elixirs, tinctures, suspensions (including nanosuspensions, microsuspensions, spray-dried dispersions), syrups, and emulsions; sublingually; bucally; parenterally, such as by subcutaneous, intravenous, intramuscular, or intrasternal injection, or infusion techniques (e.g., as sterile injectable aqueous or non-aqueous solutions or suspensions); nasally, including administration to the nasal membranes, such as by inhalation spray; topically, such as in the form of a cream or ointment; or rectally such as in the form of suppositories. They can be administered alone, but generally will be administered with a pharmaceutical carrier selected on the basis of the chosen route of administration and standard pharmaceutical practice.

The phrase “pharmaceutically acceptable carrier” as used herein means a pharmaceutically acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation, including, i.e., adjuvant, excipient or vehicle, such as diluents, preserving agents, fillers, flow regulating agents, disintegrating agents, wetting agents, emulsifying agents, suspending agents, sweetening agents, flavoring agents, perfuming agents, antibacterial agents, antifungal agents, lubricating agents and dispensing agents, depending on the nature of the mode of administration and dosage forms; and not injurious to the patient.

The term “pharmaceutical composition” means a composition comprising a compound of the invention in combination with at least one additional pharmaceutically acceptable carrier.

Pharmaceutically acceptable carriers are formulated according to a number of factors well within the purview of those of ordinary skill in the art. These include, without limitation: the type and nature of the active agent being formulated; the subject to which the agent-containing composition is to be administered: the intended route of administration of the composition; and the therapeutic indication being targeted. Pharmaceutically acceptable carriers include both aqueous and non-aqueous liquid media, as well as a variety of solid and semi-solid dosage forms. Such carriers can include a number of different ingredients and additives in addition to the active agent, such additional ingredients being included in the formulation for a variety of reasons, e.g., stabilization of the active agent, binders, etc., well known to those of ordinary skill in the art. Descriptions of suitable pharmaceutically acceptable carriers, and factors involved in their selection, are found in a variety of readily available sources such as, for example, Allen, L. V. Jr. et al. Remington: The Science and Practice of Pharmacy (2 Volumes), 22nd Edition (2012), Pharmaceutical Press.

The dosage regimen for the compounds of the present invention will, of course, vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment: the route of administration, the renal and hepatic function of the patient, and the effect desired.

By way of general guidance, the daily oral dosage of each active ingredient, when used for the indicated effects, will range between about 0.001 to about 5000 mg per day, preferably between about 0.01 to about 1000 mg per day, and most preferably between about 0.1 to about 250 mg per day. Intravenously, the most preferred doses will range from about 0.01 to about 10 mg/kg/minute during a constant rate infusion. Compounds of this invention may be administered in a single daily dose, or the total daily dosage may be administered in divided doses of two, three, or four times daily.

The compounds are typically administered in admixture with suitable pharmaceutical diluents, excipients, or carriers (collectively referred to herein as pharmaceutical carriers) suitably selected with respect to the intended form of administration. e.g., oral tablets, capsules, elixirs, and syrups, and consistent with conventional pharmaceutical practices.

Dosage forms (pharmaceutical compositions) suitable for administration may contain from about 1 milligram to about 2000 milligrams of active ingredient per dosage unit. In these pharmaceutical compositions the active ingredient will ordinarily be present in an amount of about 0.1-95% by weight based on the total weight of the composition.

A typical capsule for oral administration contains at least one of the compounds of the present invention (250 mg), lactose (75 mg), and magnesium stearate (15 mg). The mixture is passed through a 60 mesh sieve and packed into a No. L gelatin capsule.

A typical injectable preparation is produced by aseptically placing at least one of the compounds of the present invention (250 mg) into a vial, aseptically freeze-drying and sealing. For use, the contents of the vial are mixed with 2 mL of physiological saline, to produce an injectable preparation.

The present invention includes within its scope pharmaceutical compositions comprising, as an active ingredient, a therapeutically effective amount of at least one of the compounds of the present invention, alone or in combination with a pharmaceutical carrier. Optionally, compounds of the present invention can be used alone, in combination with other compounds of the invention, or in combination with one or more other therapeutic agent(s), e.g., an anticancer agent or other pharmaceutically active material.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of this invention may be varied so as to obtain an amount of the active ingredient which is effective to achieve the therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the therapeutic effect and gradually increase the dosage until the effect is achieved.

In general, a suitable daily dose of a compound of the invention will be that amount of the compound which is the lowest dose effective to produce a therapeutic effect. Such an effective dose will generally depend upon the factors described above. Generally, oral, intravenous, intracerebroventricular and subcutaneous doses of the compounds of this invention for a patient will range from about 0.01 to about 50 mg per kilogram of body weight per day.

If desired, the effective daily dose of the active compound may be administered as two, three, four, five, six or more sub-doses administered separately at appropriate intervals throughout the day, optionally, in unit dosage forms. In certain aspects of the invention, dosing is one administration per day.

While it is possible for a compound of the present invention to be administered alone, it is preferable to administer the compound as a pharmaceutical formulation (composition).

The above other therapeutic agents, when employed in combination with the compounds of the present invention, may be used, for example, in those amounts indicated in the Physicians' Desk Reference (PDR) or as otherwise determined by one of ordinary skill in the art. In the methods of the present invention, such other therapeutic agent(s) may be administered prior to, simultaneously with, or following the administration of the inventive compounds.

### Methods of Preparation

The compounds of the present invention may be synthesized by many methods available to those skilled in the art of organic chemistry. General synthetic schemes for preparing compounds of the present invention are described below. These schemes are illustrative and are not meant to limit the possible techniques one skilled in the art may use to prepare the compounds disclosed herein. Different methods to prepare the compounds of the present invention will be evident to those skilled in the art. Examples of compounds of the present invention prepared by methods described in the general schemes are given in the Examples section set out hereinafter. Preparation of homochiral examples may be carried out by techniques known to one skilled in the art. For example, homochiral compounds may be prepared by separation of racemic products or diastereomers by chiral phase preparative HPLC. Alternatively, the example compounds may be prepared by methods known to give enantiomerically or diastereomerically enriched products.

The reactions and techniques described in this section are performed in solvents appropriate to the reagents and materials employed and are suitable for the transformations being effected. Also, in the description of the synthetic methods given below, it is to be understood that all proposed reaction conditions, including choice of solvent, reaction atmosphere, reaction temperature, duration of the experiment and work up procedures, are chosen to be the conditions standard for that reaction, which should be readily recognized by one skilled in the art. It is understood by one skilled in the art of organic synthesis that the functionality present on various portions of the molecule must be compatible with the reagents and reactions proposed. Such restrictions to the substituents that are compatible with the reaction conditions will be readily apparent to one skilled in the art, with alternatives required when incompatible substituents are present. This will sometimes require a judgment to modify the order of the synthetic steps or to select one particular process scheme over another in order to obtain a compound of the invention. It will also be recognized that another major consideration in the planning of any synthetic route in this field is the judicious choice of a protecting group used for protection of reactive functional groups present in the compounds described in this invention. An authoritative account describing the many alternatives to the trained practitioner is Wuts and Greene, Greene's Protective Groups in Organic Synthesis, Fourth Edition, Wiley and Sons (2007).

Methodologies that can be employed in the syntheses of intermediates useful in the preparation of examples of the current invention are shown in the scheme below.

### EXAMPLES

The following examples illustrate the particular and preferred embodiments of the present invention and do not limit the scope of the present invention. Chemical abbreviations and symbols as well as scientific abbreviations and symbols have their usual and customary meanings unless otherwise specified. Additional abbreviations employed in the Examples and elsewhere in this application are defined above. Common intermediates are generally useful for the preparation of more than one Example and are identified sequentially (e.g., Intermediate 1, Intermediate 2, etc.) and are abbreviated as Int. 1 or I1, Int. 2 or I2, etc. Compounds of the Examples are identified by the example and step in which they were prepared (e.g., “1-A” denotes the Example 1, step A), or by the example only where the compound is the title compound of the example (for example, “1” denotes the title compound of Example 1). In some instances alternate preparations of intermediates or examples are described. Frequently chemists skilled in the art of synthesis may devise alternative preparations which may be desirable based on one or more considerations such as shorter reaction time, less expensive starting materials, ease of operation or isolation, improved yield, amenable to catalysis, avoidance of toxic reagents, accessibility of specialized instrumentation, and decreased number of linear steps, etc. The intent of describing alternative preparations is to further enable the preparation of the examples of this invention. In some instances some functional groups in the outlined examples and claims may be replaced by well-known bioisosteric replacements known in the art, for example, replacement of a carboxylic acid group with a tetrazole or a phosphate moiety. ¹H NMR data collected in deuterated dimethyl sulfoxide used water suppression in the data processing. The reported spectra are uncorrected for the effects of water suppression. Protons adjacent to the water suppression frequency of 3.35 ppm exhibit diminished signal intensity.

### Abbreviations

Ac acetyl
anhyd. anhydrous
aq. aqueous
Bu butyl
DMF dimethylformamide
DMSO dimethyl sulfoxide
EDTA ethylenediaminetetraacetic acid
Et ethyl
EtOH ethanol
h, hours or hrs hour(s)
HCl hydrochloric acid
HPLC high pressure liquid chromatography
LC liquid chromatography
LCMS liquid chromatography-mass spectrometry
M molar
M⁺¹ (M+H)⁺
Me methyl
MeOH methanol
MHz megahertz
mins minute(s)
mM millimolar
MS mass spectrometry
n or N normal
nM nanomolar
Ph phenyl
rt or Ret time retention time
sat. saturated
TFA trifluoroacetic acid

### Example 4

### 5-(5-((cyclohexyl(methyl)amino)methyl)-1H-tetrazol-1-yl)-2-(trifluoromethyl)benzonitrile

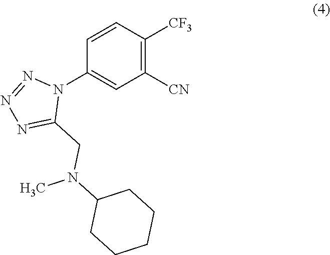 [SMILES: CN(Cc1nnnn1-c1ccc(C(F)(F)F)c(C#N)c1)C1CCCCC1]

### Step 1: Preparation of 2-chloro-N-(3-cyano-4-(trifluoromethyl)phenyl)acetamide

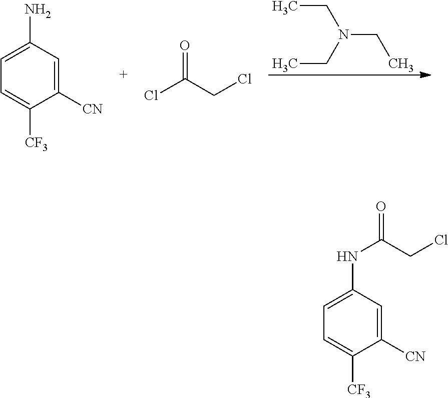

A solution of 5-amino-2-(trifluoromethyl)benzonitrile (4.8 g, 25.8 mmol) in tetrahydrofuran (103 mL) was cooled to 0° C., and triethylamine (10.78 ml, 77 mmol) and 2-chloroacetyl chloride (2.054 ml, 25.8 mmol) were added. The reaction mixture was stirred at 0° C. for 10 min. LC/MS indicated the reaction was completed. The crude reaction mixture was diluted with ethyl acetate and washed with water, brine and dried over magnesium sulfate. The solvent was removed by rotary evaporation and the residue was purified on silica gel chromatography using hexanes:ethyl acetate gradient (10:1 to 1:1) to afford 6 g of the desired product as a light yellow solid. ¹H NMR (400 MHz, chloroformn-d) δ 8.21 (dd, J=1.4, 0.6 Hz, 1H), 8.18-8.07 (m, 2H), 4.92 (s, 2H).

### Step 2: Preparation of 5-(5-(chloromethyl)-1H-tetrazol-1-yl)-2-(trifluoromethyl)benzonitrile

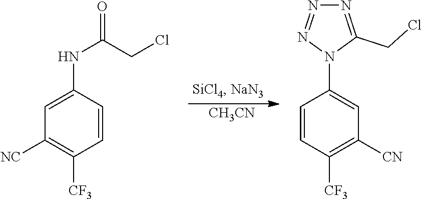 [SMILES: N#Cc1cc(NC(=O)CCl)ccc1C(F)(F)F ; "CH3CN" ; N#Cc1cc(-n2nnnc2CCl)ccc1C(F)(F)F]

In a screw top reaction vessel, 2-chloro-N-(3-cyano-4-(trifluoromethyl)phenyl) acetamide (2 g, 7.62 mmol) and sodium azide (1.980 g, 30.5 mmol) were combined in acetonitrile (40 mL), and tetrachlorosilane (3.50 mL, 30.5 mmol) was added. The reaction mixture was placed under nitrogen and stirred at 130° C. for 4 hours. The reaction was quenched with saturated sodium bicarbonate. The reaction mixture was adsorbed onto celite. Chromatography on a 40 g silica gel column with 20-100% ethyl acetate in hexane provided the title compound (575 mg, 26% yield). ¹H NMR (400 MHz, chloroform-d) δ 7.53 (d, J=8.7 Hz, 1H), 7.02 (d, J=1.9 Hz, 1H), 6.88 (br d, J=8.6 Hz, 1H), 4.26 (br s, 2H). ¹⁹F NMR (471 MHz, DMSO-d₆) δ−60.78 (s, CF₃).

### Step C: Preparation of 5-(5-((cyclohexyl(methyl)amino)methyl)-1H-tetrazol-1-yl)-2-(trifluoromethyl)benzonitrile

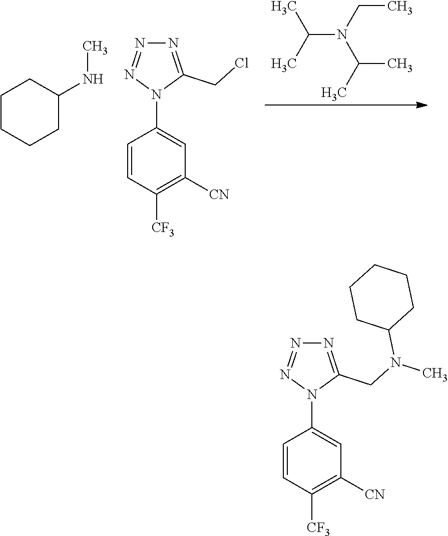

In a 100 mL round bottom flask, Hunig's base (699 μL, 4.00 mmol) and 5-(5-(chloromethyl)-1H-tetrazol-1-yl)-2-(trifluoromethyl)benzonitrile (575 mg, 2.000 mmol) were dissolved in DMF (10 mL). To this solution, N-methylcyclohexanamine (272 mg, 2.400 mmol) was added and the reaction mixture was stirred at room temperature overnight. The reaction mixture was poured into 200 mL of ethyl acetate and extracted 5 times with 25 mL water and once with 20 mL brine. The organic portion was adsorbed onto celite and chromatographed on a 24 g silica gel cartridge using a 20-100% gradient of ethyl acetate in hexanes. Fractions consistent with the desired product were combined and the solvent was removed to give the title compound (570 mg, 78% yield) as an oil. The oil was agitated under hexanes and formed a white solid. ¹H NMR (400 MHz, chloroform-d) δ 8.81 (d, J=1.6 Hz, 1H), 8.35 (dd, J=8.6, 1.3 Hz, 1H), 8.04 (d, J=8.6 Hz, 1H), 3.99 (s, 2H), 2.49 (ddd, J=11.0, 7.8, 3.5 Hz, 1H), 2.27 (s, 3H), 1.91-1.79 (m, 4H), 1.69 (br d. J=12.9 Hz, 1H), 1.42-1.21 (m, 5H), 1.20-1.06 (m, 1H). LC/MS 1.1 min., M⁺¹:364.9.

The Examples 1-30 in the table were prepared according to the general procedure disclosed in Example 4 by substituting the desired aniline into Step 1 (RAr—NH₂) and the desired secondary amine (R′R″NH) into Step 3.

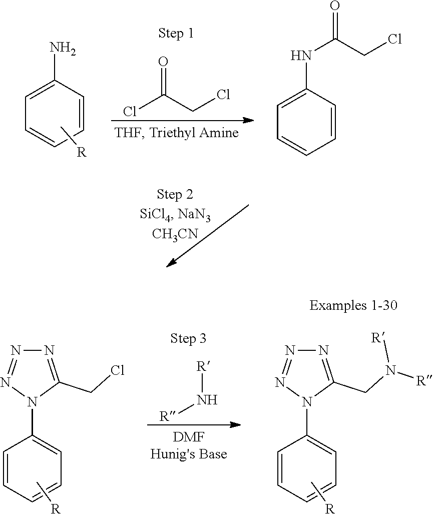

| Ex. | | LCMS | Ret | |
|---|---|---|---|---|
| No. | Structure | Method | Time min. | M + H |
| 1 | 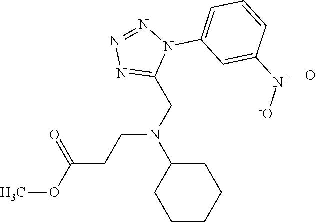 | BB | 1.5 | 389.2 |
| 2 | 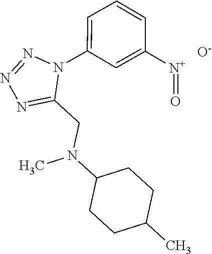 | BB | 2.2 | 331.1 |
| 3 | 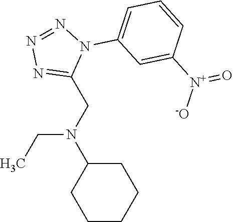 | BB | 1.2 | 331.1 |
| 4 | 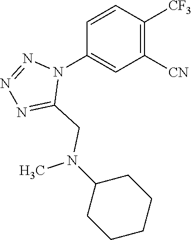 [SMILES: CN(Cc1nnnn1-c1ccc(C(F)(F)F)c(C#N)c1)C1CCCCC1] | AB | 1.1 | 364.9 |
| 5 | 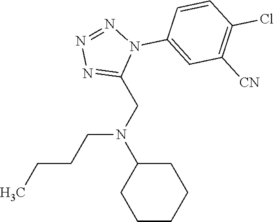 [SMILES: CCCCN(Cc1nnnn1-c1ccc(Cl)c(C#N)c1)C1CCCCC1] | A | 2.629 | 373.2 |
| 6 | 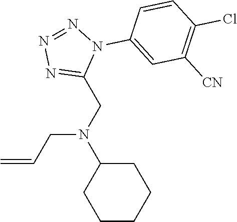 [SMILES: C=CCN(Cc1nnnn1-c1ccc(Cl)c(C#N)c1)C1CCCCC1] | A | 2.395 | 357.2 |
| 7 | 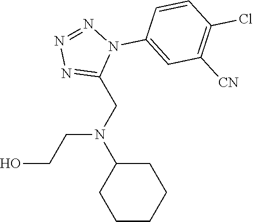 [SMILES: N#Cc1cc(-n2nnnc2CN(CCO)C2CCCCC2)ccc1Cl] | B | 1.87 | 361.2 |
| 8 | 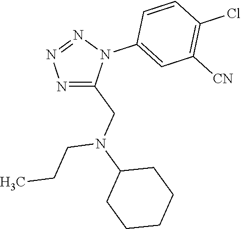 [SMILES: CCCN(Cc1nnnn1-c1ccc(Cl)c(C#N)c1)C1CCCCC1] | B | 1.815 | 359.2 |
| 9 | 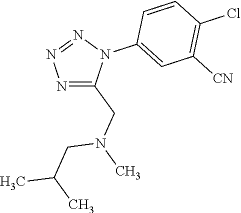 [SMILES: CC(C)CN(C)Cc1nnnn1-c1ccc(Cl)c(C#N)c1] | B | 2.242 | 305.1 |
| 10 | 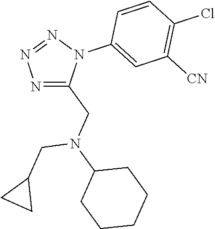 [SMILES: N#Cc1cc(-n2nnnc2CN(CC2CC2)C2CCCCC2)ccc1Cl] | B | 1.913 | 371.2 |
| 11 | 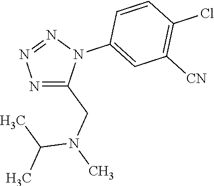 [SMILES: CC(C)N(C)Cc1nnnn1-c1ccc(Cl)c(C#N)c1] | B | 1.484 | 291.1 |
| 12 | 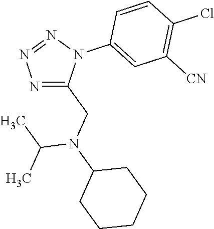 [SMILES: CC(C)N(Cc1nnnn1-c1ccc(Cl)c(C#N)c1)C1CCCCC1] | A | 2.499 | 359.2 |
| 13 | 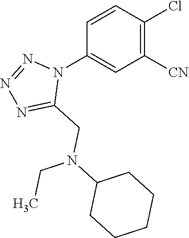 [SMILES: CCN(Cc1nnnn1-c1ccc(Cl)c(C#N)c1)C1CCCCC1] | A | 2.355 | 345.2 |
| 14 | 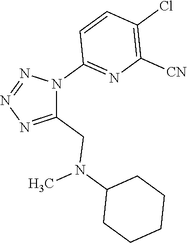 [SMILES: CN(Cc1nnnn1-c1ccc(Cl)c(C#N)n1)C1CCCCC1] | A | 2.023 | 332.1 |
| 15 | 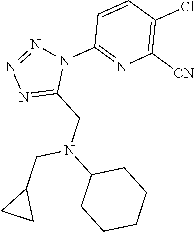 [SMILES: N#Cc1nc(-n2nnnc2CN(CC2CC2)C2CCCCC2)ccc1Cl] | A | 2.376 | 372.2 |
| 16 | 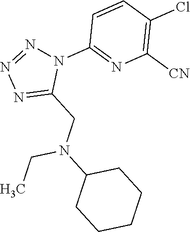 [SMILES: CCN(Cc1nnnn1-c1ccc(Cl)c(C#N)n1)C1CCCCC1] | A | 2.216 | 346.2 |
| 17 | 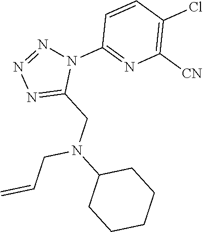 [SMILES: C=CCN(Cc1nnnn1-c1ccc(Cl)c(C#N)n1)C1CCCCC1] | B | 1.551 | 438.2 |
| 18 | 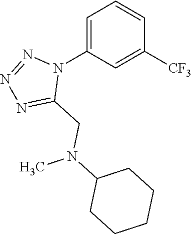 [SMILES: CN(Cc1nnnn1-c1cccc(C(F)(F)F)c1)C1CCCCC1] | AA | 2.29 | 340.2 |
| 19 | 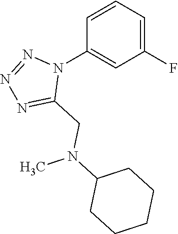 [SMILES: CN(Cc1nnnn1-c1cccc(F)c1)C1CCCCC1] | AA | 2.06 | 290.2 |
| 20 | 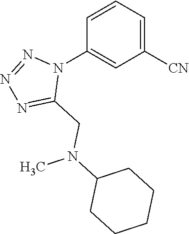 [SMILES: CN(Cc1nnnn1-c1cccc(C#N)c1)C1CCCCC1] | AA | 1.91 | 297.1 |
| 21 | 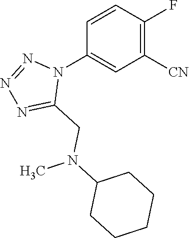 [SMILES: CN(Cc1nnnn1-c1ccc(F)c(C#N)c1)C1CCCCC1] | AA | 1.97 | 315.2 |
| 22 | 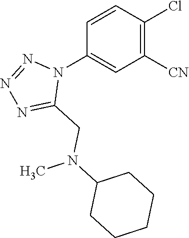 [SMILES: CN(Cc1nnnn1-c1ccc(Cl)c(C#N)c1)C1CCCCC1] | AA | 2.09 | 331.1 |
| 23 | 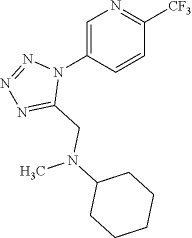 [SMILES: CN(Cc1nnnn1-c1ccc(C(F)(F)F)nc1)C1CCCCC1] | AA | 2.11 | 341.1 |
| 24 | 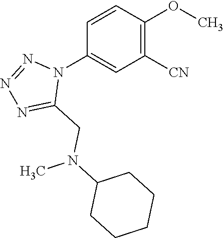 [SMILES: COc1ccc(-n2nnnc2CN(C)C2CCCCC2)cc1C#N] | AA | 1.89 | 327.0 |
| 25 | 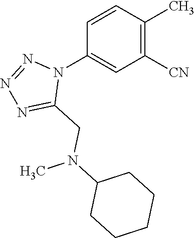 [SMILES: Cc1ccc(-n2nnnc2CN(C)C2CCCCC2)cc1C#N] | AA | 2.00 | 311.0 |
| 26 | 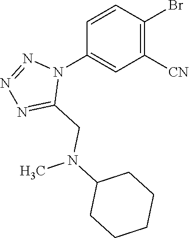 [SMILES: CN(Cc1nnnn1-c1ccc(Br)c(C#N)c1)C1CCCCC1] | AA | 2.11 | 375.1 |
| 27 | 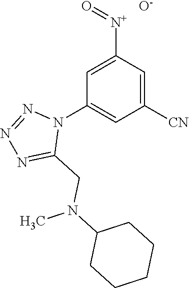 | AA | 2.09 | 342.0 |
| 28 | 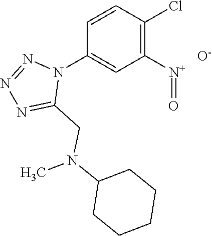 | AA | 2.19 | 351.1 |
| 29 | 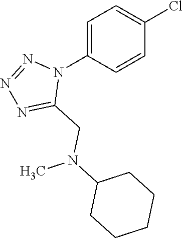 [SMILES: CN(Cc1nnnn1-c1ccc(Cl)cc1)C1CCCCC1] | AA | 2.18 | 306.1 |
| 30 | 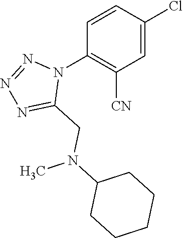 [SMILES: CN(Cc1nnnn1-c1ccc(Cl)cc1C#N)C1CCCCC1] | AA | 2.09 | 331.1 |
| 31 | 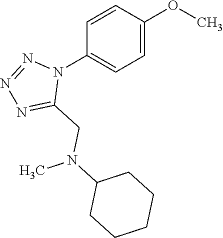 [SMILES: COc1ccc(-n2nnnc2CN(C)C2CCCCC2)cc1] | C | 2 | 302.2 |
| 32 | 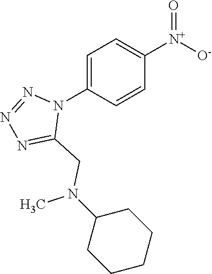 [SMILES: CN(Cc1nnnn1-c1ccc([N+](=O)[O-])cc1)C1CCCCC1] | BB | 1.0 | 317.1 |
| 33 | 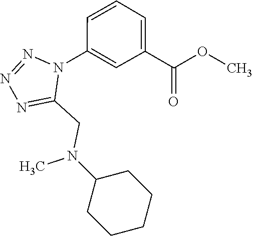 [SMILES: COC(=O)c1cccc(-n2nnnc2CN(C)C2CCCCC2)c1] | AA | 2.0 | 330.1 |
| 34 | 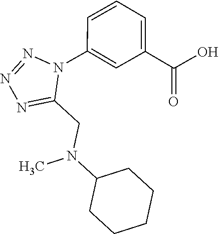 [SMILES: CN(Cc1nnnn1-c1cccc(C(=O)O)c1)C1CCCCC1] | AA | 1.1 | 315.9 |
| 35 | 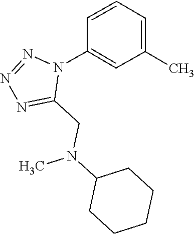 [SMILES: Cc1cccc(-n2nnnc2CN(C)C2CCCCC2)c1] | BB | 1.24 | 286.2 |
| 36 | 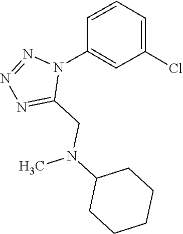 [SMILES: CN(Cc1nnnn1-c1cccc(Cl)c1)C1CCCCC1] | BB | 1.27 | 306.1 |
| 37 | 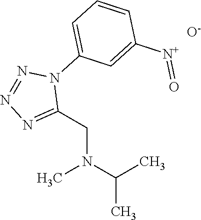 | AA | 1.7 | 344.1 |
| 38 | 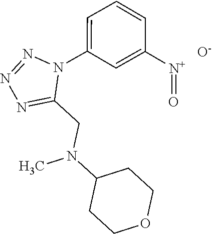 | AA | 1.4 | 319.1 |
| 39 | 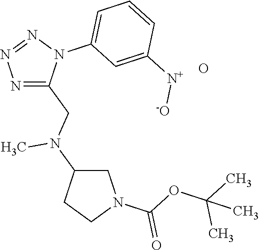 | AA | 1.8 | 426.0 |
| 40 | 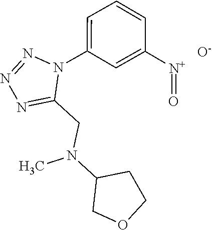 | BB | 0.98 | 305.1 |
| 41 | 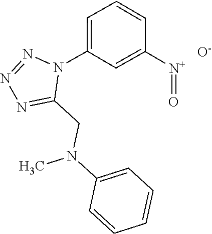 | BB | 1.75 | 311.1 |
| 42 | 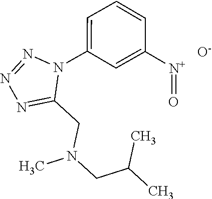 | AA | 1.92 | 291.1 |
| 43 | 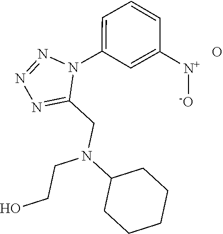 | AA | 1.69 | 347.3 |
| 44 | 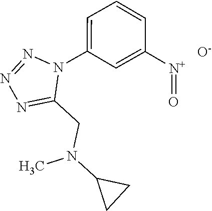 | BB | 1.09 | 274.9 |
| 45 | 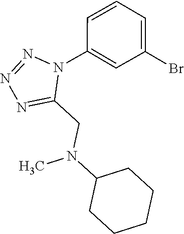 [SMILES: CN(Cc1nnnn1-c1cccc(Br)c1)C1CCCCC1] | BB | 1.3 | 350.0 |
| 46 | 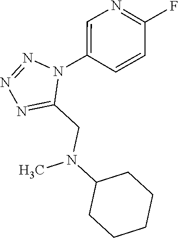 [SMILES: CN(Cc1nnnn1-c1ccc(F)nc1)C1CCCCC1] | AA | 1.82 | 291.1 |
| 47 | 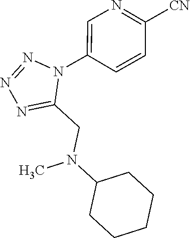 [SMILES: CN(Cc1nnnn1-c1ccc(C#N)nc1)C1CCCCC1] | BB | 1.02 | 298.1 |
| 48 | 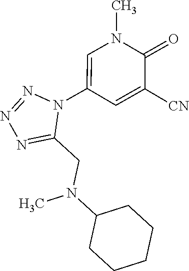 [SMILES: CN(Cc1nnnn1-c1cc(C#N)c(=O)n(C)c1)C1CCCCC1] | AA | 1.54 | 328.2 |
| 49 | 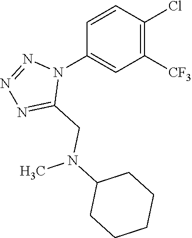 [SMILES: CN(Cc1nnnn1-c1ccc(Cl)c(C(F)(F)F)c1)C1CCCCC1] | AA | 2.43 | 374.0 |
| 50 | 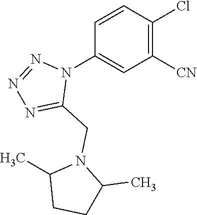 [SMILES: CC1CCC(C)N1Cc1nnnn1-c1ccc(Cl)c(C#N)c1] | A | 1.9 | 317.4 |
| 51 | 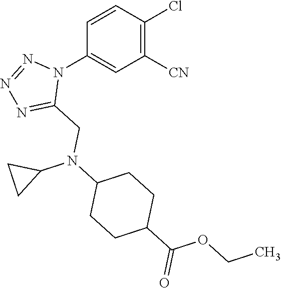 [SMILES: CCOC(=O)C1CCC(N(Cc2nnnn2-c2ccc(Cl)c(C#N)c2)C2CC2)CC1] | B | 2.3 | 429.2 |
| | 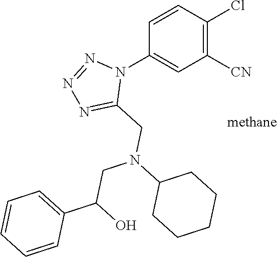 [SMILES: N#Cc1cc(-n2nnnc2CN(CC(O)c2ccccc2)C2CCCCC2)ccc1Cl] | B | 2.1 | 437.2 |
| 53 | 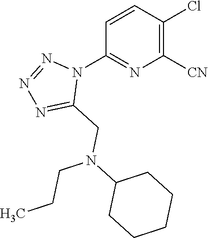 [SMILES: CCCN(Cc1nnnn1-c1ccc(Cl)c(C#N)n1)C1CCCCC1] | A | 2.4 | 360.2 |
| 54 | 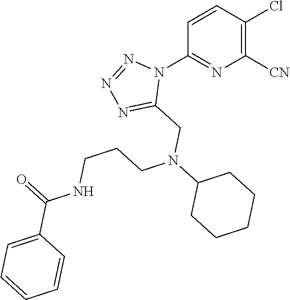 [SMILES: N#Cc1nc(-n2nnnc2CN(CCCNC(=O)c2ccccc2)C2CCCCC2)ccc1Cl] | A | 2.0 | 479.2 |
| 55 | 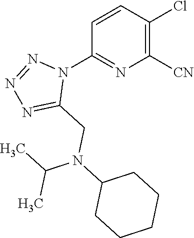 [SMILES: CC(C)N(Cc1nnnn1-c1ccc(Cl)c(C#N)n1)C1CCCCC1] | A | 2.4 | 360.2 |
| 56 | 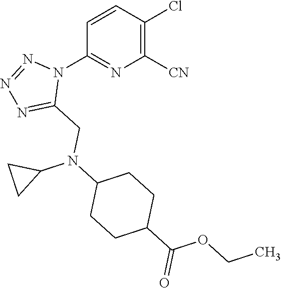 [SMILES: CCOC(=O)C1CCC(N(Cc2nnnn2-c2ccc(Cl)c(C#N)n2)C2CC2)CC1] | B | 1.5 | 430.2 |
| 57 | 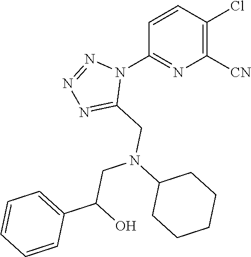 [SMILES: N#Cc1nc(-n2nnnc2CN(CC(O)c2ccccc2)C2CCCCC2)ccc1Cl] | A | 2.7 | 374.2 |
| 58 | 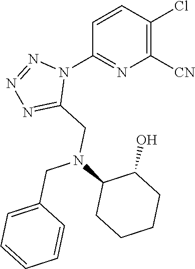 [SMILES: N#Cc1nc(-n2nnnc2CN(Cc2ccccc2)[C@@H]2CCCC[C@H]2O)ccc1Cl] | A | 2.1 | 430.2 |
| 59 | 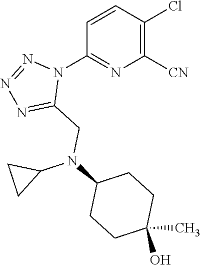 [SMILES: C[C@]1(O)CC[C@@H](N(Cc2nnnn2-c2ccc(Cl)c(C#N)n2)C2CC2)CC1] | A | 1.7 | 388.1 |
| 60 | 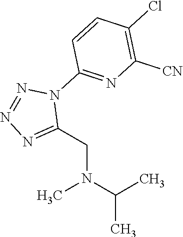 [SMILES: CC(C)N(C)Cc1nnnn1-c1ccc(Cl)c(C#N)n1] | A | 1.6 | 292.1 |
| 61 | 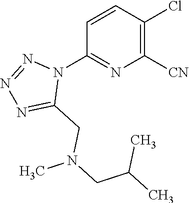 [SMILES: CC(C)CN(C)Cc1nnnn1-c1ccc(Cl)c(C#N)n1] | A | 2.0 | 306.1 |
| 62 | 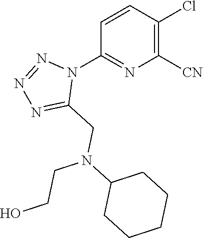 [SMILES: N#Cc1nc(-n2nnnc2CN(CCO)C2CCCCC2)ccc1Cl] | A | 1.8 | 362.2 |
| 63 | 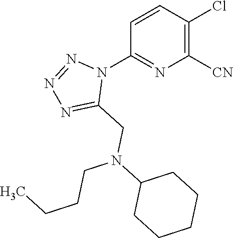 [SMILES: CCCCN(Cc1nnnn1-c1ccc(Cl)c(C#N)n1)C1CCCCC1] | A | 2.5 | 374.2 |

The following analytical LCMS method were used to characterize the compounds.

Method A: Waters XBridge BEH XP C18 (50×2.1 mm) 2.5 μm; Mobile Phase A: 5:95 acetonitrile:water with 10 mM NH₄OAc; Mobile Phase B. 95:5 acetonitrile:water with 10 mM NH₄OAc; Temperature: 50° C.; Gradient: 0-100% B over 3 minutes, Flow:

Method B: Column: Waters XBridge BEH XP C18 (50×2.1 mm) 2.5 μm; Mobile Phase A: 5:95 acetonitrile:water with 0.1% TFA; Mobile Phase B: 95:5 acetonitrile:water with 0.1% TFA; Temperature: 50° C.; Gradient: 0-100% B over 3 minutes; Flow: 1.1 mL/min.

Method AA: conditions: Column: Waters XBridge C18, 2.1 mm×50 mm, 1.7 μm particles; Mobile Phase A: 5:95 acetonitrile:water with 10 mM ammonium acetate; Mobile Phase B: 95:5 acetonitrile:water with 10 mM ammonium acetate; Temperature: 50° C.: Gradient: 0% B to 100% B over 3 min, then a 0.75 min hold at 100% B; Flow: 1 mL/min; Detection: MS and UV (220 nm).

Method AB: Gradient: Start % B: 0, Final % B: 100, Gradient Time: 1.80 min, Stop Time: 2.00 min, Flow Rate: 1.0 mL/min., Wavelength 1: 220 nm, Solvent A:0.05% TFA in CH₃CN:Water (5:95), Solvent B: 0.05% TFA in CH3CN:Water (95:5), Column: Acquity BEH C18 1.7 μm 2.1×50 mm.

Method BB: Column: Waters XBridge C18, 2.1 mm×50 mm, 1.7 μm particles; Mobile Phase A: 5:95 acetonitrile:water with 0.1% trifluoroacetic acid; Mobile Phase B: 95:5 acetonitrile:water with 0.1% trifluoroacetic acid; Temperature: 50° C.; Gradient: 0% B to 100% B over 3 min, then a 0.75 min hold at 100% B; Flow: 1 mL/min; Detection: MS and UV (220 nm).

Method C: Column: Waters BEH C18, 2.0×50 mm, 1.7-μm particles: Mobile Phase A: 5:95 acetonitrile:water with 10 mM ammonium acetate, Mobile Phase B: 95:5 acetonitrile:water with 10 mM ammonium acetate; Temperature: 50° C.: Gradient: 0-100% B over 3 minutes, then a 0.5-minute hold at 100% B: Flow: 1.0 mL/min; Detection: UV at 220 nm.

### Biological Assays

DGK alpha ADPGLO Full Length IC₅₀ (uM)
DGK alpha ADPGLO (Near Full Length) IC₅₀ (uM)

### In Vitro DGK Inhibition Assays

The DGKα (Full Length and Near Full Length) ADP Glo assays were performed using an extruded liposome prep at either 5% DAG or 10% DAG. The Enzymatic reactions were carried out in 50 mM MOPS pH 7.5, 100 mM NaCl, 10 mM MgCl₂, 1 μM CaCl₂, and 1 mM DTT (assay buffer). The lipid substrate concentrations were 1.9 mM PS, either 0.25 mM DAG (5% liposome prep) or 0.5 mM DAG (10% liposome prep), and 2.7 mM PC for the extruded liposome reactions. The reactions were carried out at 150 μM ATP. The enzyme concentrations for the DGKα(Near Full Length) and DGKα(FL) were 5 nM.

The compound inhibition studies were carried out as follows: 25 nL droplets of each test compound (11 point, 3-fold dilution series for each compound) solubilized in DMSO were transferred to wells of a white 1536 well plate (Corning 3725). A 5 mL enzyme/substrate (substrate being the 5× or 10× liposome preparation) solution at 2× final reaction concentrations was prepared by combining 5 ml of the extruded liposome preparation with 10 nM (2× final) DGKα(Near Full Length) or DGKα(Full Length) prepared as described below) and incubated at room temperature for 10 minutes. Next, 1 μL of the 2× enzyme/substrate solution was added to wells containing the test compound and reactions were initiated with the addition of 1 μL 300 uM ATP. Substrate only was added to DMSO only wells for background measurements. The reactions were allowed to proceed for 1 hr, after which 2 μL Glo Reagent (Promega V9101) was added and incubated for 40 minutes. Next, 4 μL Kinase Detection Reagent was added and incubated for 30 minutes. Luminescence was recorded using an EnVision microplate reader. The percent inhibition was calculated from the ATP conversion generated by no enzyme control reactions for 100% inhibition and vehicle-only reactions for 0% inhibition. The compounds were evaluated at 11 concentrations to determine IC₅₀.

### 2× Liposome Preparation (5% DAG)

The lipid composition was 5 mol % DAG (Avanti 800811O), 40 mol % PS (Avanti 840035P), and 55 mol % PC (Avanti 850457) at a total lipid concentration of 7-8 mg/mL for the liposome solution. The PC, DAG, and PS were dissolved in chloroform, combined, and dried in vacuo to a thin film. The lipids were hydrated to 20 mM in 50 mM MOPS pH 7.5, 100 mM NaCl, 5 mM MgCl₂, and were freeze-thawed five times. The lipid suspension was extruded through a 100 nm polycarbonate filter 10-12 times. Dynamic light scattering was carried out to confirm liposome size (50-60 nm radius). The liposome preparation was stored at 4° C. for as long as four weeks.

### 2× Liposome Preparation (10% DAG)

The lipid composition was 9.7 mol %, 1 mM DAG (Avanti 800811O), 37.3 mol %, 3.8 mM PS (Avanti 840035P), and 53 mol %, 5.4 mM PC (Avanti 850457) at a total lipid concentration of 7-8 mg/mL (10.2 mM Total Lipids) for the 2× liposome solution. To prepare these liposomes, the PC, DAG, and PS were dissolved in chloroform, combined, and dried in vacuum to a thin film. The lipids were hydrated to 10 mM in 50 mM MOPS pH 7.5, 100 mM NaCl, 1 μM CaCl₂), 10 mM MgCl₂, 1 mM DTT, and were freeze-thawed five times. The lipid suspension was extruded through a 100 nrm polycarbonate filter eleven times. Dynamic light scattering was carried out to confirm liposome size (50-60 nm radius). The liposome preparation was stored at ambient temperature for as long as four weeks.

### Cloning and Expression of Full Length Human DGKα in Baculovirus.

The DNA fragment (Ref Seq_NP_958852.1) encoding full length DGKα was codon optimized for insect cell expression and gene synthesized at GenScript, USA Inc. (Piscataway, NJ) and cloned into a modified pFastBac1 vector (Invitrogen, Carlsbad, CA) as NdeI-XhoI fragment.

The baculoviruses expressing hDGKα-TVMV-was generated using the Bac-to-Bac baculovirus expression system (Invitrogen) according to the manufacturer's protocol.

The expression scale up of hDGKα-TVMV-His was carried out in Sf9 cell (Expression System, Davis, CA) cultures grown to a density of 2×10⁶ cells/mL in ESF921 insect medium (Expression Systems) and infected with virus stock at 1:200 virus/cell ratio. Cultures were maintained at a volume of 800 mL, 130 rpm and grown for 65 hrs at 27° C. post-infection. The infected cell cultures were harvested by centrifugation at 2000 rpm for 20 min 4° C. in a SORVALL® RC12BP centrifuge. The cell pellets were stored at −70° C. until purification

### Purification of Full Length Human DGK-α

Full length human DGKα (SEQ ID No. 2) was expressed as TVMV cleavable C-terminal Hexa His tag and purified from SF9 baculovirus-infected insect paste. The cell pellet was resuspended at a 1:6 mass ratio of cells to lysis buffer (50 mM HEPES, pH 7.4, 0.3 M NaCl, 5% glycerol, 1 mM TCEP), supplemented with 20 mM imidazole and Complete EDTA free Protease Inhibitor tablets and Benzonase. The cells were lysed by using nitrogen decompression method with a nitrogen bomb (Parr Instruments), and pressurizing the suspended cells at 300 psi for 30 min at 4° C. The lysate was clarified by ultracentrifugation at 100,000×g for 45 min. Purification steps were carried using an ÅKTA Purifier Plus system. The clarified supernatant was applied to a 5 mL HisTrap FF crude Nickel Affinity column, washed to baseline and eluted with 50 mM HEPES, pH 7.4, 0.3 M NaCl, 5% glycerol, 1 mM TCEP with 500 mM Imidazole. Fractions containing the target protein were pooled, concentrated and further purified on a HiLoad 26/600 Superdex 200 pg Size Exclusion chromatography pre-equilibrated in 50 mM HEPES. pH 7.4, 0.2 M NaCl, 5% glycerol, 1 mM TCEP. Fractions containing the target protein were pooled and diluted 4 fold with Dilution buffer [50 mM HEPES, pH 7.4, 5% glycerol, 1 mM TCEP] to reduce NaCl concentration from 0.2 M to 0.05 M. Diluted sizing pool was applied to a 5 mL HiTrap Q Sepharose FF Anion Exchange column, washed to baseline and eluted with 0% to 100% Elution buffer [50 mM HEPES, pH 7.4, 1.0 M NaCl, 5% glycerol, 1 mM TCEP] in 10 column volume. Fractions containing the target protein were pooled concentrated between 2-5 mg/ml, flash frozen in liquid nitrogen and stored at −80° C. in 0.1 mg aliquots. A final purity of 80% was achieved, with yield of 0.5-2 mg per L cell culture.

### Cloning and Expression of Human DGKα (Near Full Length) in Baculovirus.

The DNA fragment (Ref Seq_NP_958852.1) encoding the near full length DGKα (hDGKα(S9-S727) was codon optimized for insect cell expression and gene synthesized at GenScript, USA Inc. (Piscataway. NJ) and cloned into a modified pFastBac1 vector (Invitrogen, Carlsbad, CA) as NdeI-XhoI fragment.

The baculoviruses expressing hDGKα(S9-S727)-TVMV-was generated using the Bac-to-Bac baculovirus expression system (Invitrogen) according to the manufacturer's protocol.

The expression scale up of hDGKα(S9-S727)-TVMV-His was carried out in Sf9 cell (Expression System, Davis, CA) cultures grown to a density of 2×10⁶ cells/mL in ESF921 insect medium (Expression Systems) and infected with virus stock at 1:200 virus/cell ratio. Cultures were maintained at a volume of 800 mL, 130 rpm and grown for 65 hrs at 27° C. post-infection. The infected cell cultures were harvested by centrifugation at 2000 rpm for 20 min 4° C. in a SORVALL@, RC12BP centrifuge. The cell pellets were stored at −70° C. until purification

### Purification of Near Full Length Human DGKα(S9-S727)

Near full length human DGKα (SEQ ID No. 2) containing amino acids 9-727 was expressed as TVMV cleavable C-terminal Hexa His tag and purified from SF9 baculovirus-infected insect paste. The cell pellet was resuspended at a 1:6 mass ratio of cells to lysis buffer (50 mM HEPES, pH 7.3, 0.3 M NaCl, 5% glycerol, 1 mM TCEP), supplemented with 20 mM imidazole and Complete EDTA free Protease Inhibitor tablets and Benzonase. The cells were lysed by using nitrogen decompression method with a nitrogen bomb (Parr Instruments), and pressurizing the suspended cells at 300 psi for 30 min at 4° C. The lysate was clarified by ultracentrifugation at 100,000×g for 45 min. Purification steps were carried using an ÅKTA Purifier Plus system. The clarified supernatant was applied to a 5 mL HisTrap FF crude Nickel Affinity column, washed to baseline and eluted with 50 mM HEPES, pH 7.3, 0.3 M NaCl, 5% glycerol, 1 mM TCEP with 500 mM Imidazole. Fractions containing the target protein were pooled, concentrated and further purified on a HiLoad 26/600 Superdex 200 pg Size Exclusion chromatography pre-equilibrated in 50 mM HEPES, pH 7.3, 0.3 M NaCl, 5% glycerol, 1 mM TCEP. A final purity of 80% was achieved, with yield of 4 mg per L cell culture. Purified near full length hDGKα was concentrated to >1 mg/ml, flash frozen in liquid nitrogen and stored at −80° C. in 0.5 mg aliquots. Liquid chromatography/mass spectrometry confirmed the identity of the protein, and verified it to have acetylation modification with cleavage of the N-terminal methionine. The association state as determined size exclusion chromatography with inline multiangle light scattering (SEC-MALS) data demonstrate that the purified protein predominantly exists as monomer with <0.5% high molecular weight aggregates.

### In Vitro DGKα Translocation Assays

Jurkat cells were transduced with lentiviral constructs overexpressing DGKα-YFP (N-terminal tagged), selected by puromycin, and were FACs sorted to establish stable lines expressing each isoform (Jurkat-DGKα-YFP=BXA-212700-01-001, Jurkat-DGKz-YFP=BXA-212701-01-001). Cell lines were maintained in growth media (RPMI supplemented with 10% FBS and pen/strep). All translocation assays were run in assay media (RPMI supplemented with 10% FBS and no antibiotics).

For translocation assays cells were plated onto Perkin Elmer CellCarrier-384 Ultra Microplates (Perkin Elmer cat #6057500) at a density of 30,000 cells/well, in a volume of 40 μL of assay media. Compounds were then transferred to the cells by an Echo Acoustic Liquid Handler (Echo 655). 40 nL droplets of each testcompound (top concentration 10 mM with 11 point, 3-old dilution series for each compound) solubilized in DMSO were transferred to each well, resulting in a dose response treatment with a top concentration of 10 μM. Treated cells were then incubated at 37° C. for 1 hour. Cells were then fixed with 4% formaldehyde (Thermo cat #28906) for 15 minutes, centrifuged briefly to increase cell retention (250 rpm for 5 minutes), and then washed 3× with 50 μL PBS. Cells were then stained with Hoechst, and washed 2× with 50 ul PBS. Next, 50 μL PBS was added to each well, plates were sealed, and plates were imaged on an Opera Phenix High Content screening system. Imaging was done with the 40× water objective. The YFP channel was set at 100% power for 500 ins, 10 regions/well were imaged and analyzed. Data analysis was done with Columbus Image Analysis software (Perk-in Elmer).

TABLE A

| | Full Length | Near Full Length | DGKalpha |
|---|---|---|---|
| | DGKalpha ADPGLO | DGKalpha ADPGLO | Translocation |
| Ex. No. | IC₅₀ (μM) | IC₅₀ (μM) | EC₅₀ (μM) |
| 2 | 2.7 | 1.2 | 0.30 |
| 3 | 0.64 | 0.37 | 4.2 |
| 4 | 0.027 | 0.28 | 0.82 |
| 5 | 1.1 | 1.4 | 1.2 |
| 6 | 0.11 | 0.10 | 0.19 |
| 7 | 4.7 | 3.4 | >10 |
| 8 | 1.5 | 2.1 | 0.35 |
| 9 | 5.3 | 3.2 | 1.2 |
| 10 | 0.25 | 0.24 | 0.63 |
| 11 | 8.8 | 8.6 | >10 |
| 12 | 7.9 | 11 | 1.7 |
| 13 | 0.09 | 0.08 | 0.12 |
| 14 | 2.0 | 1.1 | 1.8 |
| 15 | 3.1 | 4.3 | 2.1 |
| 16 | 2.7 | 2.6 | >10 |
| 17 | 1.2 | 1.6 | 0.47 |
| 18 | 9.7 | 11 | >5.0 |
| 19 | 5.2 | 5.8 | >5.0 |
| 20 | 3.9 | 3.7 | >10 |
| 21 | 3.7 | 3.1 | 1.2 |
| 22 | 0.08 | 0.090 | 0.06 |
| 23 | 6.5 | 3.6 | >5 |
| 24 | 0.90 | 0.86 | 1.7 |
| 25 | 0.14 | 0.41 | 0.35 |
| 26 | 0.02 | 0.040 | 0.020 |
| 27 | — | 0.48 | 0.49 |
| 28 | 0.06 | 0.12 | — |
| 29 | 3.5 | 1.7 | 0.57 |
| 30 | — | — | >10 |
| 31 | 56 | — | — |
| 32 | 13 | — | — |
| 33 | >240 | — | — |
| 34 | >81 | — | — |
| 35 | 50 | — | — |
| 36 | 11 | — | — |
| 37 | 36 | — | — |
| 38 | 15 | — | — |
| 39 | 35 | — | — |
| 40 | 55 | — | — |
| 41 | 32 | — | — |
| 42 | 32 | — | — |
| 43 | 72 | — | — |
| 44 | 180 | — | — |
| 45 | 12 | — | — |
| 46 | 16 | — | — |
| 47 | 84 | — | — |
| 48 | 56 | — | — |
| 49 | 17 | — | — |
| 50 | 54 | — | — |
| 51 | 13 | — | — |
| 52 | 11 | — | — |
| 53 | 15 | — | — |
| 54 | 70 | — | — |
| 55 | 46 | — | — |
| 56 | 11 | — | — |
| 57 | 120 | — | — |
| 58 | 60 | — | — |
| 59 | 180 | — | — |
| 60 | 67 | — | — |
| 61 | 24 | — | — |
| 62 | 110 | — | — |
| 63 | 15 | — | — |

The compounds of the present invention possess activity as an inhibitor(s) of one or both of the DGKζ and DGKζ enzymes, and therefore, may be used in the treatment of diseases associated with the inhibition of DGKα and DGKζ activity.

| Nucleotide sequence encoding hDGKα-(M1-S735)-Ct-TVMV-His: | | |
|---|---|---|
| (SEQ ID NO: 1) | | |
| 1 | ATGGCCAAGG AGAGGGGCCT AATAAGCCCC AGTGATTTTG CCCAGCTGCA | |
| 51 | AAAATACATG GAATACTCCA CCAAAAAGGT CAGTGATGTC CTAAAGCTCT | |
| 101 | TCGAGGATGG CGAGATGGCT AAATATGTCC AAGGAGATGC CATTGGGTAC | |
| 151 | GAGGGATTCC AGCAATTCCT GAAAATCTAT CTCGAAGTGG ATAATGTTCC | |
| 201 | CAGACACCTA AGCCTGGCAC TGTTTCAATC CTTTGAGACT GGTCACTGCT | |
| 251 | TAAATGAGAC AAATGTGACA AAAGATGTGG TGTGTCTCAA TGATGTTTCC | |
| 301 | TGCTACTTTT CCCTTCTGGA GGGTGGTCGG CCAGAAGACA AGTTAGAATT | |
| 351 | CACCTTCAAG CTGTACGACA CGGACAGAAA TGGGATCCTG GACAGCTCAG | |
| 401 | AAGTGGACAA AATTATCCTA CAGATGATGC GAGTGGCTGA ATACCTGGAT | |
| 451 | TGGGATGTGT CTGAGCTGAG GCCGATTCTT CAGGAGATGA TGAAAGAGAT | |
| 501 | TGACTATGAT GGCAGTGGCT CTGTCTCTCA AGCTGAGTGG GTCCGGGCTG | |
| 551 | GGGCCACCAC CGTGCCACTG CTAGTGCTGC TGGGTCTGGA GATGACTCTG | |
| 601 | AAGGACGACG GACAGCACAT GTGGAGGCCC AAGAGGTTCC CCAGACCAGT | |
| 651 | CTACTGCAAT CTGTGCGAGT CAAGCATTGG TCTTGGCAAA CAGGGACTGA | |
| 701 | GCTGTAACCT CTGTAAGTAC ACTGTTCACG ACCAGTGTGC CATGAAAGCC | |
| 751 | CTGCCTTGTG AAGTCAGCAC CTATGCCAAG TCTCGGAAGG ACATTGGTGT | |
| 801 | CCAATCACAT GTGTGGGTGC GAGGAGGCTG TGAGTCCGGG CGCTGCGACC | |
| 851 | GCTGTCAGAA AAAGATCCGG ATCTACCACA GTCTGACCGG GCTGCATTGT | |
| 901 | GTATGGTGCC ACCTAGAGAT CCACGATGAC TGCCTGCAAG CGGTGGGCCA | |
| 951 | TGAGTGTGAC TGTGGGCTGC TCCGGGATCA CATCCTGCCT CCATCTTCCA | |
| 1001 | TCTATCCCAG TGTCCTGGCC TCTGGACCGG ATCGTAAAAA TAGCAAAACA | |
| 1051 | AGCCAGAAGA CCATGGATGA TTTAAATTTG AGCACCTCTG AGGCTCTGCG | |
| 1101 | GATTGACCCT GTTCCTAACA CCCACCCACT TCTCGTCTTT GTCAATCCTA | |
| 1151 | AGAGTGGCGG GAAGCAGGGG CAGAGGGTGC TCTGGAAGTT CCAGTATATA | |
| 1201 | TTAAACCCTC GACAGGTGTT CAACCTCCTA AAGGATGGTC CTGAGATAGG | |
| 1251 | GCTCCGATTA TTCAAGGATG TTCCTGATAG CCGGATTTTG GTGTGTGGTG | |
| 1301 | GAGACGGCAC AGTAGGCTGG ATTCTAGAGA CCATTGACAA AGCTAACTTG | |
| 1351 | CCAGTTTTGC CTCCTGTTGC TGTGTTGCCC CTGGGTACTG GAAATGATCT | |
| 1401 | GGCTCGATGC CTAAGATGGG GAGGAGGTTA TGAAGGACAG AATCTGGCAA | |
| 1451 | AGATCCTCAA GGATTTAGAG ATGAGTAAAG TGGTACATAT GGATCGATGG | |
| 1501 | TCTGTGGAGG TGATACCTCA ACAAACTGAA GAAAAAAGTG ACCCAGTCCC | |
| 1551 | CTTTCAAATC ATCAATAACT ACTTCTCTAT TGGCGTGGAT GCCTCTATTG | |
| 1601 | CTCATCGATT CCACATCATG CGAGAGAAAT ATCCGGAGAA GTTCAACAGC | |
| 1651 | AGAATGAAGA ACAAGCTATG GTACTTCGAA TTTGCCACAT CTGAATCCAT | |
| 1701 | CTTCTCAACA TGCAAAAAGC TGGAGGAGTC TTTGACAGTT GAGATCTGTG | |
| 1751 | GGAAACCGCT GGATCTGAGC AACCTGTCCC TAGAAGGCAT CGCAGTGCTA | |
| 1801 | AACATCCCTA GCATGCATGG TGGCTCCAAC CTCTGGGGTG ATACCAGGAG | |
| 1851 | ACCCCATGGG GATATCTATG GGATCAACCA GGCCTTAGGT GCTACAGCTA | |
| 1901 | AAGTCATCAC CGACCCTGAT ATCCTGAAAA CCTGTGTACC AGACCTAAGT | |
| 1951 | GACAAGAGAC TGGAAGTGGT TGGGCTGGAG GGTGCAATTG AGATGGGCCA | |
| 2001 | AATCTATACC AAGCTCAAGA ATGCTGGACG TCGGCTGGCC AAGTGCTCTG | |
| 2051 | AGATCACCTT CCACACCACA AAAACCCTTC CCATGCAAAT TGACGGAGAA | |
| 2101 | CCCTGGATGC AGACGCCCTG TACAATCAAG ATCACCCACA AGAACCAGAT | |
| 2151 | GCCCATGCTC ATGGGCCCAC CCCCCCGCTC CACCAATTTC TTTGGCTTCT | |
| 2201 | TGAGCGGATC CTCGGAGACA GTGCGGTTTC AGGGACACCA CCACCATCAC | |
| 2251 | CACTGA | |
| Amino acid sequence of hDGKα-(M1-S735)-Ct-TVMV-His: | | |
| (SEQ ID NO: 2) | | |
| 0001 | MAKERGLISP SDFAQLQKYM EYSTKKVSDV LKLFEDGEMA KYVQGDAIGY EGFQQFLKIY | 0060 |
| 0061 | LEVDNVPRHL SLALFQSFET GHCLNETNVT KDVVCLNDVS CYFSLLEGGR PEDKLRFTFK | 0120 |
| 0121 | LYDTDRNGIL DSSEVDKIIL QMMRVAEYLD WDVSELRPIL QEMMKEIDYD GSGSVSQAEW | 0180 |
| 0181 | VRAGATTVPL LVLLGLEMTL KDDGQHMWRP KRFPRPVYCN LCESSIGLGK QGLSCNLCKY | 0240 |
| 0241 | TVHDQCAMKA LPCEVSTYAK SRKDIGVOSH VWVRGGCESG RCDRCQKKIR IYHSLTGLHC | 0300 |
| 0301 | VWCHLEIHDD CLQAVGHECD CGLLRDHILP PSSIYPSVLA SGPDRKNSKT SQKTMDDLNL | 0360 |
| 0361 | STSEALRIDP VPNTHPLLVF VNPKSGGKQG QRVLWKFQYI LNPRQVENLL KDGPEIGLRL | 0420 |
| 0421 | FKDVPDSRIL VCGGDGTVGW ILETIDKANL PVLPPVAVLP LGTGNDLARC LRWGGGYEGQ | 0480 |
| 0481 | NLAKILKDLE MSKVVHMDRW SVEVIPQQTE EKSDPVPFQI INNYFSIGVD ASIAHRFHIM | 0540 |
| 0541 | REKYPEKENS RMKNKLWYFE FATSESIFST CKKLEESLTV EICGKPLDLS NLSLEGIAVL | 0600 |
| 0601 | NIPSMHGGSN LWGDTRRPHG DIYGINQALG ATAKVITDPD ILKTCVPDLS DKRLEVVGLE | 0660 |
| 0661 | GAIEMGQIYT KLKNAGRRLA KCSEITFHTT KTLPMQIDGE PWMQTPCTIK ITHKNQMPML | 0720 |
| 0721 | MGPPPRSTNF FGFLSGSSET VRFQGHHHHH H | 0751 |
| Nucleotide sequence encoding hDGKζ-(M1-A928)-transcript variant-2 | | |
| Ct-TVMV-His: | | |
| (SEQ ID NO: 3) | | |
| 1 | ATGGAGCCGC GGGACGGTAG CCCCGAGGCC CGGAGCAGCG ACTCCGAGTC | |
| 51 | GGCTTCCGCC TCGTCCAGCG GCTCCGAGCG CGACGCCGGT CCCGAGCCGG | |
| 101 | ACAAGGCGCC GCGGCGACTC AACAAGCGGC GCTTCCCGGG GCTGCGGCTC | |
| 151 | TTCGGGCACA GGAAAGCCAT CACGAAGTCG GGCCTCCAGC ACCTGGCCCC | |
| 201 | CCCTCCGCCC ACCCCTGGGG CCCCGTGCAG CGAGTCAGAG CGGCAGATCC | |
| 251 | GGAGTACAGT GGACTGGAGC GAGTCAGCGA CATATGGGGA GCACATCTGG | |
| 301 | TTCGAGACCA ACGTGTCCGG GGACTTCTGC TACGTTGGGG AGCAGTACTG | |
| 351 | TGTAGCCAGG ATGCTGCAGA AGTCAGTGTC TCGAAGAAAG TGCGCAGCCT | |
| 401 | GCAAGATTGT GGTGCACACG CCCTGCATCG AGCAGCTGGA GAAGATAAAT | |
| 451 | TTCCGCTGTA AGCCGTCCTT CCGTGAATCA GGCTCCAGGA ATGTCCGCGA | |
| 501 | GCCAACCTTT GTACGGCACC ACTGGGTACA CAGACGACGC CAGGACGGCA | |
| 551 | AGTGTCGGCA CTGTGGGAAG GGATTCCAGC AGAAGTTCAC CTTCCACAGC | |
| 601 | AAGGAGATTG TGGCCATCAG CTGCTCGTGG TGCAAGCAGG CATACCACAG | |
| 651 | CAAGGTGTCC TGCTTCATGC TGCAGCAGAT CGAGGAGCCG TGCTCGCTGG | |
| 701 | GGGTCCACGC AGCCGTGGTC ATCCCGCCCA CCTGGATCCT CCGCGCCCGG | |
| 751 | AGGCCCCAGA ATACTCTGAA AGCAAGCAAG AAGAAGAAGA GGGCATCCTT | |
| 801 | CAAGAGGAAG TCCAGCAAGA AAGGGCCTGA GGAGGGCCGC TGGAGACCCT | |
| 851 | TCATCATCAG GCCCACCCCC TCCCCGCTCA TGAAGCCCCT GCTGGTGTTT | |
| 901 | GTGAACCCCA AGAGTGGGGG CAACCAGGGT GCAAAGATCA TCCAGTCTTT | |
| 951 | CCTCTGGTAT CTCAATCCCC GACAAGTCTT CGACCTGAGC CAGGGAGGGC | |
| 1001 | CCAAGGAGGC GCTGGAGATG TACCGCAAAG TGCACAACCT GCGGATCCTG | |
| 1051 | GCGTGCGGGG GCGACGGCAC GGTGGGCTGG ATCCTCTCCA CCCTGGACCA | |
| 1101 | GCTACGCCTG AAGCCGCCAC CCCCTGTTGC CATCCTGCCC CTGGGTACTG | |
| 1151 | GCAACGACTT GGCCCGAACC CTCAACTGGG GTGGGGGCTA CACAGATGAG | |
| 1201 | CCTGTGTCCA AGATCCTCTC CCACGTGGAG GAGGGGAACG TGGTACAGCT | |
| 1251 | GGACCGCTGG GACCTCCACG CTGAGCCCAA CCCCGAGGCA GGGCCTGAGG | |
| 1301 | ACCGAGATGA AGGCGCCACC GACCGGTTGC CCCTGGATGT CTTCAACAAC | |
| 1351 | TACTTCAGCC TGGGCTTTGA CGCCCACGTC ACCCTGGAGT TCCACGAGTC | |
| 1401 | TCGAGAGGCC AACCCAGAGA AATTCAACAG CCGCTTTCGG AATAAGATGT | |
| 1451 | TCTACGCCGG GACAGCTTTC TCTGACTTCC TGATGGGCAG CTCCAAGGAC | |
| 1501 | CTGGCCAAGC ACATCCGAGT GGTGTGTGAT GGAATGGACT TGACTCCCAA | |
| 1551 | GATCCAGGAC CTGAAACCCC AGTGTGTTGT TTTCCTGAAC ATCCCCAGGT | |
| 1601 | ACTGTGCGGG CACCATGCCC TGGGGCCACC CTGGGGAGCA CCACGACTTT | |
| 1651 | GAGCCCCAGC GGCATGACGA CGGCTACCTC GAGGTCATTG GCTTCACCAT | |
| 1701 | GACGTCGTTG GCCGCGCTGC AGGTGGGCGG ACACGGCGAG CGGCTGACGC | |
| 1751 | AGTGTCGCGA GGTGGTGCTC ACCACATCCA AGGCCATCCC GGTGCAGGTG | |
| 1801 | GATGGCGAGC CCTGCAAGCT TGCAGCCTCA CGCATCCGCA TCGCCCTGCG | |
| 1851 | CAACCAGGCC ACCATGGTGC AGAAGGCCAA GCGGCGGAGC GCCGCCCCCC | |
| 1901 | TGCACAGCGA CCAGCAGCCG GTGCCAGAGC AGTTGCGCAT CCAGGTGAGT | |
| 1951 | CGCGTCAGCA TGCACGACTA TGAGGCCCTG CACTACGACA AGGAGCAGCT | |
| 2001 | CAAGGAGGCC TCTGTGCCGC TGGGCACTGT GGTGGTCCCA GGAGACAGTG | |
| 2051 | ACCTAGAGCT CTGCCGTGCC CACATTGAGA GACTCCAGCA GGAGCCCGAT | |
| 2101 | GGTGCTGGAG CCAAGTCCCC GACATGCCAG AAACTGTCCC CCAAGTGGTG | |
| 2151 | CTTCCTGGAC GCCACCACTG CCAGCCGCTT CTACAGGATC GACCGAGCCC | |
| 2201 | AGGAGCACCT CAACTATGTG ACTGAGATCG CACAGGATGA GATTTATATC | |
| 2251 | CTGGACCCTG AGCTGCTGGG GGCATCGGCC CGGCCTGACC TCCCAACCCC | |
| 2301 | CACTTCCCCT CTCCCCACCT CACCCTGCTC ACCCACGCCC CGGTCACTGC | |
| 2351 | AAGGGGATGC TGCACCCCCT CAAGGTGAAG AGCTGATTGA GGCTGCCAAG | |
| 2401 | AGGAACGACT TCTGTAAGCT CCAGGAGCTG CACCGAGCTG GGGGCGACCT | |
| 2451 | CATGCACCGA GACGAGCAGA GTCGCACGCT CCTGCACCAC GCAGTCAGCA | |
| 2501 | CTGGCAGCAA GGATGTGGTC CGCTACCTGC TGGACCACGC CCCCCCAGAG | |
| 2551 | ATCCTTGATG CGGTGGAGGA AAACGGGGAG ACCTGTTTGC ACCAAGCAGC | |
| 2601 | GGCCCTGGGC CAGCGCACCA TCTGCCACTA CATCGTGGAG GCCGGGGCCT | |
| 2651 | CGCTCATGAA GACAGACCAG CAGGGCGACA CTCCCCGGCA GCGGGCTGAG | |
| 2701 | AAGGCTCAGG ACACCGAGCT GGCCGCCTAC CTGGAGAACC GGCAGCACTA | |
| 2751 | CCAGATGATC CAGCGGGAGG ACCAGGAGAC GGCTGTGGGA TCCTCGGAGA | |
| 2801 | CAGTGCGGTT TCAGGGACAC CACCACCATC ACCACTGA | |
| Amino acid sequence of hDGKζ-(MI-A928)-transcript variant-2 Ct-TVMV-His: | | |
| (SEQ ID NO: 4) | | |
| 0001 | MEPRDGSPEA RSSDSESASA SSSGSERDAG PEPDKAPRRL NKRRFPGIRL FGERKAITKS | 0060 |
| 0061 | GLQHLAPPPP TPGAPCSESE RQIRSTVDWS ESATYGEHIW FETNVSGDFC YVGEQYCVAR | 0120 |
| 0121 | mLQKSVSRRK CAACKIVVHT PCIEQLEKIN FRCKPSFRES GSRNVREPTF VREHWVHRRR | 0180 |
| 0181 | QDGKCRHCGK GFQQKFTFHS KEIVAISCSW CKOAYHSKVS CEMLQQIEEP CSLGVHAAVV | 0240 |
| 0241 | IPPTWILRAR RPQNTLKASK KKKRASFKRK SSKKGPEEGR WRPFIIRPTP SPLMKPLLVF | 0300 |
| 0301 | VNPKSGGNQG AKIIQSFLWY LNPRQVEDLS QGGPKEALEM YRKVHNERIL ACGGDGTVGW | 0360 |
| 0361 | ILSTLDQLRL KPPPPVAILP LGTGNDLART LNWGGGYTDE PVSKILSHVE EGNVVQLDRW | 0420 |
| 0421 | DLHAEPNPEA GPEDRDEGAT DRLPLDVENN YESLGEDAHV TLEFHESREA NPEKENSRFR | 0480 |
| 0481 | NKMFYAGTAF SDFIMGSSKD LAKHIRVVCD GMDLTPKIQD LKPQCVVELN IPRYCAGTMP | 0540 |
| 0541 | WGHPGEHHDF EPQRHDDGYL EVIGFTMTSL AALQVGGHGE RLTQCREVVL TTSKAIPVQV | 0600 |
| 0601 | DGEPCKLAAS RIRIALRNQA TMVQKAKRRS AAPLASDQQP VPEQLRIQVS RVSMHDYEAL | 0660 |
| 0661 | HYDKEQLKEA SVPLGTVVVP GDSDLELCRA HIERLQQEPD GAGAKSPTCQ KLSPKWCFLD | 0720 |
| 0721 | ATTASRFYRI DRAQEHLNYV TEIAQDEIYI LDPELLGASA RPDLPTPTSP LPTSPCSPTP | 0780 |
| 0781 | RSLQGDAAPP QGEELIEAAK RNDFCKLQEL HRAGGDLMHR DEQSRTLLHH AVSTGSKDVV | 0840 |
| 0841 | RYLLDHAPPE ILDAVEENGE TCLHQAAALG QRTICHYIVE AGASIMKTDQ QGDTPRQRAE | 0900 |
| 0901 | KAQDTELAAY LENRQHYQMI QREDQETAVG SSETVRFQGH HHHHH | 0945 |
| Nucleotide sequence encoding MA-hDGKα-(S9-S727)-Ct-TVMV-His: | | |
| (SEQ ID NO: 5) | | |
| 0001 | ATGGCTTCCC CAAGCGACTT CGCCCAGCTG CAGAAGTACA TGGAATACAG CACCAAGAAG | 0060 |
| 0061 | GTGTCTGACG TCCTGAAGCT GTTCGAGGAC GGTGAAATGG CTAAGTACGT CCAGGGCGAC | 0120 |
| 0121 | GCTATCGGAT ACGAGGGATT CCAGCAGTTC CTGAAGATCT ACCTGGAAGT GGACAACGTC | 0180 |
| 0181 | CCCAGGCACC TGTCACTGGC TCTGTTCCAG TCCTTCGAGA CTGGCCACTG CCTGAACGAA | 0240 |
| 0241 | ACCAACGTCA CTAAGGACGT GGTCTGCCTG AACGACGTGA GCTGCTACTT CTCTCTGCTG | 0300 |
| 0301 | GAGGGTGGCA GACCAGAGGA CAAGCTGGAA TTCACCTTCA AGCTGTACGA CACTGACCGC | 0360 |
| 0361 | AACGGAATCC TGGACTCCAG CGAAGTGGAC AAGATCATCC TGCAGATGAT GCGTGTCGCT | 0420 |
| 0421 | GAGTACCTGG ACTGGGACGT GAGCGAACTG AGGCCTATCC TGCAGGAGAT GATGAAGGAA | 0480 |
| 0481 | ATCGACTACG ACGGCTCTGG ATCAGTGTCC CAGGCTGAGT GGGTCCGCGC TGGTGCTACC | 0540 |
| 0541 | ACTGTGCCAC TGCTGGTCCT GCTGGGACTG GAAATGACCC TGAAGGACGA CGGTCAGCAC | 0600 |
| 0601 | ATGTGGCGCC CAAAGCGTTT CCCCAGGCCA GTCTACTGCA ACCTGTGCGA GTCTTCAATC | 0660 |
| 0661 | GGTCTGGGCA AGCAGGGCCT GTCATGCAAC CTGTGCAAGT ACACCGTGCA CGACCAGTGC | 0720 |
| 0721 | GCTATGAAGG CCCTGCCCTG CGAGGTCTCA ACTTACGCTA AGTCCCGTAA GGACATCGGA | 0780 |
| 0781 | GTGCAGTCAC ACGTGTGGGT CAGGGGAGGT TGCGAATCCG GTAGATGCGA CCGCTGCCAG | 0840 |
| 0841 | AAGAAGATCC GTATCTACCA CTCCCTGACC GGACTGCACT GCGTCTGGTG CCACCTGGAG | 0900 |
| 0901 | ATCCACGACG ACTGCCTGCA GGCCGTGGGA CACGAATGCG ACTGCGGTCT GCTGCGTGAC | 0960 |
| 0961 | CACATCCTGC CTCCCTCCAG CATCTACCCT TCAGTCCTGG CTTCCGGTCC CGACAGGAAG | 1020 |
| 1021 | AACAGCAAGA CCTCTCAGAA GACTATGGAC GACCTGAACC TGAGCACCTC TGAGGCCCTG | 1080 |
| 1081 | CGCATCGACC CTGTGCCCAA CACTCACCCA CTGCTGGTGT TCGTCAACCC TAAGAGCGGC | 1140 |
| 1141 | GGAAAGCAGG GTCAGAGAGT CCTGTGGAAG TTCCAGTACA TCCTGAACCC ACGCCAGGTG | 1200 |
| 1201 | TTCAACCTGC TGAAGGACGG CCCTGAGATC GGACTGAGAC TGTTCAAGGA CGTGCCCGAC | 1260 |
| 1261 | TCTCGCATCC TCGTCTGCGG TGGCGACGGT ACTGTGGGAT GGATCCTGGA AACTATCGAC | 1320 |
| 1321 | AAGGCTAACC TGCCAGTGCT GCCACCTGTG GCTGTCCTGC CACTGGGAAC CGGTAACGAC | 1380 |
| 1381 | CTGGCTCGTT GCCTGCGTTG GGGAGGTGGC TACGAGGGAC AGAACCTGGC CAAGATCCTG | 1440 |
| 1441 | AAGGACCTGG AAATGAGCAA GGTGGTCCAC ATGGACAGAT GGTCTGTGGA GGTCATCCCA | 1500 |
| 1501 | CAGCAGACTG AGGAAAAGTC AGACCCAGTC CCTTTCCAGA TCATCAACAA CTACTTCAGC | 1560 |
| 1561 | ATCGGTGTGG ACGCTTCTAT CGCCCACAGA TTCCACATCA TGCGCGAGAA GTACCCTGAA | 1620 |
| 1621 | AAGTTCAACT CCCGCATGAA GAACAAGCTG TGGTACTTCG AGTTCGCTAC CTCAGAATCC | 1680 |
| 1681 | ATCTTCTCAA CTTGCAAGAA GCTGGAGGAA TCCCTGACCG TCGAGATCTG CGGCAAGCCT | 1740 |
| 1741 | CTGGACCTGT CAAACCTGTC CCTGGAAGGC ATCGCTGTGC TGAACATCCC AAGCATGCAC | 1800 |
| 1801 | GGAGGTTCTA ACCTCTGGGG CGACACTAGG AGGCCTCACG GTGACATCTA CGGCATCAAC | 1860 |
| 1861 | CAGGCCCTGG GAGCTACCGC CAAGGTCATC ACTGACCCCG ACATCCTGAA GACCTGCGTG | 1920 |
| 1921 | CCAGACCTGA GCGACAAGCG TCTGGAGGTG GTCGGACTGG AGGGTGCCAT CGAAATGGGC | 1980 |
| 1981 | CAGATCTACA CTAAGCTGAA GAACGCTGGA AGGAGACTGG CCAAGTGCTC TGAGATCACC | 2040 |
| 2041 | TTCCACACCA CTAAGACTCT GCCTATGCAG ATCGACGGTG AACCCTGGAT GCAGACCCCA | 2100 |
| 2101 | TGCACTATCA AGATCACCCA CAAGAACCAG ATGCCCATGC TGATGGGTCC TCCTCCTCGC | 2160 |
| 2161 | TCTGGATCTT CAGAAACTGT GAGGTTCCAG GGCCACCACC ACCACCACCA CTGA | 2214 |
| Amino acid sequence of MA-hDGKa-(S9-S727)-Ct-TVMV-His: | | |
| (SEQ ID NO: 6) | | |
| 0001 | MASPSDFAQL QKYMEYSTKK VSDVLKLFED GEMAKYVOGD AIGYEGFQQF LKIYLEVDNV | 0060 |
| 0061 | PRHLSLALFQ SFETGHCINE TNVTKDVVCL NDVSCYFSLL EGGRPEDKLE FTFKLYDTDR | 0120 |
| 0121 | NGILDSSEVD KIILQMMRVA EYLQWDVSEL RPILQEMMKE IDYDGSGSVS QAEWVRAGAT | 0180 |
| 0181 | TVPLLVLLGL EMTLKDDGQH MWRPKRFPRP VYCNLCESSI GLGKQGLSCN LCKYTVHDQC | 0240 |
| 0241 | AMKALPCEVS TYAKSRKDIG VQSHVWVRGG CESGRCDRCQ KKIRIYHSLT GLHCVWCHLE | 0300 |
| 0301 | IHDDCLQAVG HECDCGLLRD HILPPSSIYP SVLASGPDRK NSKTSQKTMD DLNLSTSEAL | 0360 |
| 0361 | RIDPVPNTHP LLVFVNPKSG GKQGQRVLWK FQYILNPRQV FNLLKDGPEI GLRLFKDVPD | 0420 |
| 0421 | SRILVCGGDG TVGWILETID KANLPVLPPV AVLPLGTGND LARCLRWGGG YEGQNLAKIL | 0480 |
| 0481 | KDLEMSKVVH MDRWSVEVIP QQTEEKSDPV PFQIINNYFS IGVDASIAHR FHIMREKYPE | 0540 |
| 0541 | KENSRMKNKL WYFEFATSES IFSTCKKLEE SLTVEICGKP LDLSNLSLEG IAVLNIPSMH | 0600 |
| 0601 | GGSNLWGDIR RPHGDIYGIN QALGATAKVI TDPDILKTCV PDLSDKRLEV VGLEGAIEMG | 0660 |
| 0661 | QIYTKLKNAG RRLAKCSEIT FHTTKTLPMQ IDGEPWMQTP CTIKITHKNQ MPMLMGPPPR | 0720 |
| 0721 | SGSSETVRFQ GHHHHHH | 0737 |

What is claimed is:

1. A compound of Formula (I): 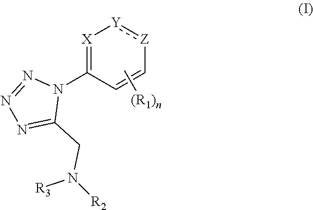
or a salt thereof, wherein:
(i) X is N, CH, or CR₁; Y is N, CH, or CR₁; and Z is CH or CR₁; provided that zero or one of X and Y is N; or
(ii) X is CH or CR₁; Y is NRᵢₐ; and Z is C(═O); [symbol]
represents either a single bond when Z is C(═O) or a double bond when Z is CH or CR₁;
each R₁ is independently F, Cl, Br, —CN, C₁₋₃ alkyl, C₁₋₂ fluoroalkyl, C₁₋₃ alkoxy, C₁₋₂ fluoroalkoxy, —C(O)OH, —C(O)O(C₁₋₃ alkyl), or —NO₂;
R₁ₐ is hydrogen or —CH₃;
R₂ is C₃₋₄ alkyl or a cyclic group selected from C₃₋₆ cycloalkyl, oxetanyl, tetrahydrofuranyl, tetrahydropyranyl, azetidinyl, pyrrolidinyl, piperidinyl, piperazinyl, bicyclo[2.2.1]heptanyl, bicyclo[3.1.0]hexanyl, bicyclo[4.1.0]heptanyl, bicyclo[3.1.1]heptanyl, bicyclo[3.2.1]octanyl, bicyclo[2.2.2]octanyl, spiro[2.5]octanyl, cubanyl, phenyl, and a 5- to 6-membered heteroaryl having 1 to 3 heteroatoms selected from N, O, and S, wherein each cyclic group is substituted with zero to 3 R₂ₐ;
each R₂ₐ is independently F, Cl, Br, —OH, —CN, C₁₋₃ alkyl, C₁₋₂ fluoroalkyl, or —C(O)(C₁₋₂ alkyl);
R₃ is C₁₋₆ alkyl, C₁₋₃ fluoroalkyl, C₁₋₄ hydroxyalkyl, C₃₋₆ cycloalkyl, —CH₂(C₃₋₆cycloalkyl), —CH₂(phenyl), —CRₓRₓCRₓ(OH)(phenyl), —CRₓRₓCRₓ=CRₓRₓ, —(CRₓRₓ)₁₋₂C(O)O(C₁₋₂ alkyl), or —(CRₓRₓ)₁₋₃NRₓC(O)(phenyl);
each Rₓ is independently hydrogen or —CH₃; and
n is zero, 1, 2, or 3;
with the provisos:
(i) the moiety 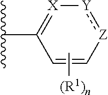
is not 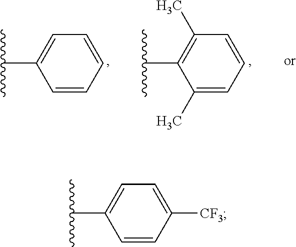
and
(ii) wherein the compound of Formula (I) is not

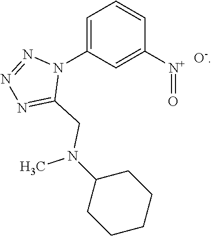

2. The compound according to claim 1 or a salt thereof, wherein:
each R₁ is independently F, Cl, Br, —CN, C₁₋₃ alkyl, —CHF₂, —CF₃, —OCH₃, —OCF₃, —C(O)OH, —C(O)O(C₁₋₂ alkyl), or —NO₂;
R₂ is C₃₋₄ alkyl or a cyclic group selected from C₃₋₆ cycloalkyl, tetrahydrofuranyl, tetrahydropyranyl, pyrrolidinyl, and phenyl, each substituted with zero to 2 R₂;
each R₂ₐ is independently —OH, —CN, —CH₃, —CH₂F, —CHF₂, —CF₃, or —C(O)OCH₂CH₃;
R₃ is C₁₋₄ alkyl, C₁₋₂ fluoroalkyl, C₁₋₃ hydroxyalkyl, C₃₋₄ cycloalkyl, —CH₂(C₃₋₄cycloalkyl), —CH₂(phenyl), —CH₂CH(OH)(phenyl), —CH₂CH═CH₂, —CH₂CH₂C(O)O(C₁₋₂ alkyl), or —CH₂CH₂CH₂NHC(O)(phenyl);
n is zero, 1, 2, or 3.

3. The compound according to claim 1 or a salt thereof, wherein:
each R₁ is independently F, Cl, Br, —CN, —CH₃, —CF₃, —OCH₃, —C(O)OH, —C(O)OCH₃, or —NO₂;
R₂ is —CH(CH₃)₂, —CH₂CH(CH₃)₂, cyclopropyl, cyclohexyl substituted with zero to 2 R₂ₐ, tetrahydrofuranyl, tetrahydropyranyl, pyrrolidinyl substituted with —C(O)OCH₂CH₃, or phenyl;
each R₂ₐ is independently —OH, —CH₃, or —C(O)OCH₂CH₃;
R₃ is —CH₃, —CH₂CH₃, —CH₂CH₂CH₃, —CH(CH₃)₂, —CH₂CH₂CH₂CH₃, —CH₂CH₂OH, cyclopropyl, —CH₂(cyclopropyl), —CH₂(phenyl), —CH₂CH(OH)(phenyl), —CH₂CH═CH₂, —CH₂CH₂C(O)OCH₃, or —CH₂CH₂CH₂NHC(O)(phenyl);
n is 1 or 2.

4. The compound according to claim 1 or a salt thereof, wherein:
X is N, CH, or CR₁;
Y is N, CH, or CR₁;
Z is CH or CR₁; and [symbol]
represents a double bond;
provided that zero or one of X and Y is N.

5. The compound according to claim 1 or a salt thereof, having the structure of Formula (IIa):

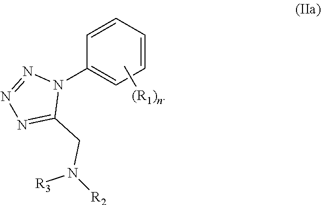

6. The compound according to claim 1 or a salt thereof, having the structure of Formula (IIb) or Formula (IIc):

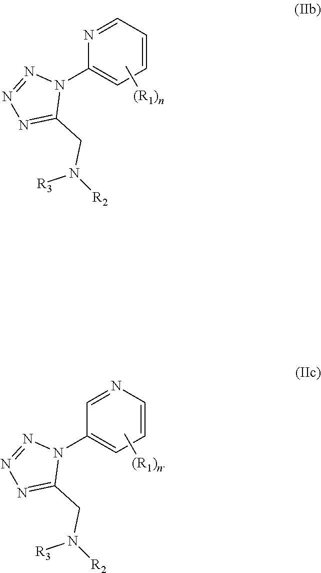

7. The compound according to claim 1 or a salt thereof, wherein R₂ is cyclohexyl substituted with zero to 2 R₂ₐ.

8. The compound according to claim 1 or a salt thereof, wherein R₂ is —CH(CH₃)₂ or —CH₂C(CH₃)₂.

9. The compound according to claim 1 or a salt thereof, wherein R₃ is C₁₋₄ alkyl.

10. The compound according to claim 1 or a salt thereof, having a structure selected from:

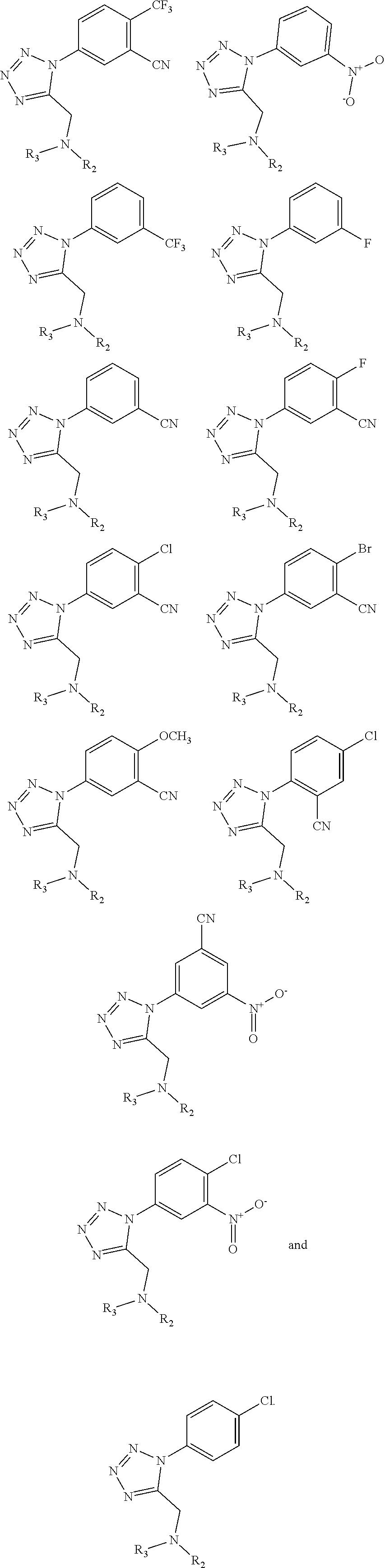

11. The compound according to claim 1 or a salt thereof, having a structure selected from:

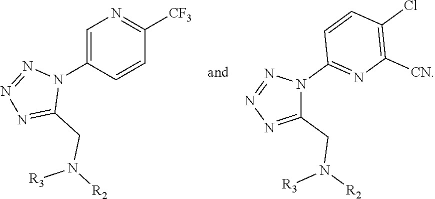

12. The compound according to claim 1 or a salt thereof, wherein said compound is:
methyl 3-(cyclohexyl((1-(3-nitrophenyl)-1H-tetrazol-5-yl)methyl)amino)propanoate (1);
N,4-dimethyl-N-((1-(3-nitrophenyl)-1H-tetrazol-5-yl)methyl)cyclohexan-1-amine (2);
N-ethyl-N-((1-(3-nitrophenyl)-1H-tetrazol-5-yl)methyl)cyclohexanamine (3);
5-(5-((cyclohexyl(methyl)amino)methyl)-1H-tetrazol-1-yl)-2-(trifluoromethyl)benzonitrile (4);
5-(5-((butyl(cyclohexyl)amino)methyl)-1H-tetrazol-1-yl)-2-chlorobenzonitrile (5);
5-(5-((allyl(cyclohexyl)amino)methyl)-1H-tetrazol-1-yl)-2-chlorobenzonitrile (6);
2-chloro-5-(5-((cyclohexyl(2-hydroxyethyl)amino)methyl)-1H-tetrazol-1-yl)benzonitrile (7);
2-chloro-5-(5-((cyclohexyl(propyl)amino)methyl)-1H-tetrazol-1-yl)benzonitrile (8);
2-chloro-5-(5-((isobutyl(methyl)amino)methyl)-1H-tetrazol-1-yl)benzonitrile (9);
2-chloro-5-(5-((cyclohexyl(cyclopropylmethyl)amino)methyl)-1H-tetrazol-1-yl)benzonitrile (10);
2-chloro-5-(5-((isopropyl(methyl)amino)methyl)-1H-tetrazol-1-yl)benzonitrile (11);
2-chloro-5-(5-((cyclohexyl(isopropyl)amino)methyl)-1H-tetrazol-1-yl)benzonitrile (12);
2-chloro-5-(5-((cyclohexyl(ethyl)amino)methyl)-1H-tetrazol-1-yl)benzonitrile (13);
3-chloro-6-(5-((cyclohexyl(methyl)amino)methyl)-1H-tetrazol-1-yl)picolinonitrile (14);
3-chloro-6-(5-((cyclohexyl(cyclopropylmethyl)amino)methyl)-1H-tetrazol-1-yl) picolinonitrile (15);
3-chloro-6-(5-((cyclohexyl(ethyl)amino)methyl)-1H-tetrazol-1-yl)picolinonitrile (16);
6-(5-((allyl(cyclohexyl)amino)methyl)-1H-tetrazol-1-yl)-3-chloropicolinonitrile (17);
N-methyl-N-((1-(3-(trifluoromethyl)phenyl)-1H-tetrazol-5-yl)methyl) cyclohexanamine (18);
N-((1-(3-fluorophenyl)-1H-tetrazol-5-yl)methyl)-N-methylcyclohexanamine (19);
3-(5-((cyclohexyl(methyl)amino)methyl)-1H-tetrazol-1-yl)benzonitrile (20);
5-(5-((cyclohexyl(methyl)amino)methyl)-1H-tetrazol-1-yl)-2-fluorobenzonitrile (21);
2-chloro-5-(5-((cyclohexyl(methyl)amino)methyl)-1H-tetrazol-1-yl)benzonitrile (22);
N-methyl-N-((1-(6-(trifluoromethyl)pyridin-3-yl)-1H-tetrazol-5-yl)methyl) cyclohexanamine (23);
5-(5-((cyclohexyl(methyl)amino)methyl)-1H-tetrazol-1-yl)-2-methoxybenzonitrile (24);
5-(5-((cyclohexyl(methyl)amino)methyl)-1H-tetrazol-1-yl)-2-methylbenzonitrile (25);
2-bromo-5-(5-((cyclohexyl(methyl)amino)methyl)-1H-tetrazol-1-yl)benzonitrile (26);
3-(5-((cyclohexyl(methyl)amino)methyl)-1H-tetrazol-1-yl)-5-nitrobenzonitrile (27);
N-((1-(4-chloro-3-nitrophenyl)-1H-tetrazol-5-yl)methyl)-N-methylcyclohexanamine (28);
N-((1-(4-chlorophenyl)-1H-tetrazol-5-yl)methyl)-N-methylcyclohexanamine (29);
5-chloro-2-(5-((cyclohexyl(methyl)amino)methyl)-1H-tetrazol-1-yl)benzonitrile (30);
N-((1-(4-methoxyphenyl)-1H-tetrazol-5-yl)methyl)-N-methylcyclohexanamine (31);
N-methyl-N-((1-(4-nitrophenyl)-1H-tetrazol-5-yl)methyl)cyclohexanamine (32);
methyl 3-(5-((cyclohexyl(methyl)amino)methyl)-1H-tetrazol-1-yl)benzoate (33);
3-(5-((cyclohexyl(methyl)amino)methyl)-1H-tetrazol-1-yl)benzoic acid (34);
N-methyl-N-((1-(m-tolyl)-1H-tetrazol-5-yl)methyl)cyclohexanamine (35);
N-((1-(3-chlorophenyl)-1H-tetrazol-5-yl)methyl)-N-methylcyclohexanamine (36);
N-methyl-N-((1-(3-nitrophenyl)-1H-tetrazol-5-yl)methyl)propan-2-amine (37);
N-methyl-N-((1-(3-nitrophenyl)-1H-tetrazol-5-yl)methyl)tetrahydro-2H-pyran-4-amine (38);
tert-butyl 3-(methyl((1-(3-nitrophenyl)-1H-tetrazol-5-yl)methyl)amino)pyrrolidine-1-carboxylate (39);
N-methyl-N-((1-(3-nitrophenyl)-1H-tetrazol-5-yl)methyl)tetrahydrofuran-3-amine (40);
N-methyl-N-((1-(3-nitrophenyl)-1H-tetrazol-5-yl)methyl)aniline (41);
N,2-dimethyl-N-((1-(3-nitrophenyl)-1H-tetrazol-5-yl)methyl)propan-1-amine (42);
2-(cyclohexyl((1-(3-nitrophenyl)-1H-tetrazol-5-yl)methyl)amino)ethan-1-ol (43);
N-methyl-N-((1-(3-nitrophenyl)-1H-tetrazol-5-yl)methyl)cyclopropanamine (44);
N-((1-(3-bromophenyl)-1H-tetrazol-5-yl)methyl)-N-methylcyclohexanamine (45);
N-((1-(6-fluoropyridin-3-yl)-1H-tetrazol-5-yl)methyl)-N-methylcyclohexanamine (46);
5-(5-((cyclohexyl(methyl)amino)methyl)-1H-tetrazol-1-yl)picolinonitrile (47);
5-(5-((cyclohexyl(methyl)amino)methyl)-1H-tetrazol-1-yl)-1-methyl-2-oxo-1,2-dihydropyridine-3-carbonitrile (48);
N-((1-(4-chloro-3-(trifluoromethyl)phenyl)-1H-tetrazol-5-yl)methyl)-N-methylcyclohexanamine (49);
ethyl 4-(((1-(4-chloro-3-cyanophenyl)-1H-tetrazol-5-yl)methylxcyclopropyl)amino) cyclohexane-1-carboxylate (51);
2-chloro-5-(5-((cyclohexyl(2-hydroxy-2-phenylethyl)amino)methyl)-1H-tetrazol-1-yl)benzonitrile (52);
3-chloro-6-(5-((cyclohexyl(propyl)amino)methyl)-1H-tetrazol-1-yl)picolinonitrile (53);
N-(3-(((1-(5-chloro-6-cyanopyridin-2-yl)-1H-tetrazol-5-yl)methylxcyclohexyl) amino)propyl)benzamide (54);
3-chloro-6-(5-((cyclohexyl(isopropyl)amino)methyl)-1H-tetrazol-1-yl)picolinonitrile (55);
ethyl 4-(((1-(5-chloro-6-cyanopyridin-2-yl)-1H-tetrazol-5-yl)methylxcyclopropyl) amino)cyclohexane-1-carboxylate (56);
3-chloro-6-(5-((cyclohexyl(2-hydroxy-2-phenylethyl)amino)methyl)-1H-tetrazol-1-yl) picolinonitrile (57);
6-(5-((benzyl((1R,2R)-2-hydroxycyclohexyl)amino)methyl)-1H-tetrazol-1-yl)-3-chloropicolinonitrile (58);
3-chloro-6-(5-((cyclopropyl((1s,4s)-4-hydroxy-4-methylcyclohexyl)amino)methyl)-1H-tetrazol-1-yl)picolinonitrile (59);
3-chloro-6-(5-((isopropyl(methyl)amino)methyl)-1H-tetrazol-1-yl)picolinonitrile (60);
3-chloro-6-(5-((isobutyl(methyl)amino)methyl)-1H-tetrazol-1-yl)picolinonitrile (61);
3-chloro-6-(5-((cyclohexyl(2-hydroxyethyl)amino)methyl)-1H-tetrazol-1-yl) picolinonitrile (62); or
6-(5-((butyl(cyclohexyl)amino)methyl)-1H-tetrazol-1-yl)-3-chloropicolinonitrile (63).

13. A pharmaceutical composition comprising a compound according to claim 1 or a pharmaceutically-acceptable salt thereof; and a pharmaceutically acceptable carrier.

14. A method for treating a disease comprising the administration to a subject in need thereof a therapeutically-effective amount of at least one compound according to claim 1 or a pharmaceutically-acceptable salt thereof, wherein said disease is cancer or viral infections.

15. The method according to claim 14, wherein said cancer is selected from cancer of the colon, pancreatic cancer, breast cancer, prostate cancer, lung cancer, ovarian cancer, cervical cancer, renal cancer, cancer of the head and neck, lymphoma, leukemia, and melanoma.

16. A method of inhibiting inhibiting activity of at least one of diacylglycerol kinase selected from diacylglycerol kinase alpha (DGKα) and diacylglycerol kinase zeta (DGKζ) comprising administering to a subject in need thereof a therapeutically effective amount of at least one compound according to claim 1.


SEQUENCE LISTING (6 sequences)

SEQ ID NO: 1 - DNA, 2256 bp, Homo sapiens
  source 1..2256: mol_type = genomic DNA
atggccaaggagaggggcctaataagccccagtgattttgcccagctgcaaaaatacatggaatactccaccaaaaaggtcagtgatgtcctaaagctcttcgaggatggcgagatggctaaatatgtccaaggagatgccattgggtacgagggattccagcaattcctgaaaatctatctcgaagtggataatgttcccagacacctaagcctggcactgtttcaatcctttgagactggtcactgcttaaatgagacaaatgtgacaaaagatgtggtgtgtctcaatgatgtttcctgctacttttcccttctggagggtggtcggccagaagacaagttagaattcaccttcaagctgtacgacacggacagaaatgggatcctggacagctcagaagtggacaaaattatcctacagatgatgcgagtggctgaatacctggattgggatgtgtctgagctgaggccgattcttcaggagatgatgaaagagattgactatgatggcagtggctctgtctctcaagctgagtgggtccgggctggggccaccaccgtgccactgctagtgctgctgggtctggagatgactctgaaggacgacggacagcacatgtggaggcccaagaggttccccagaccagtctactgcaatctgtgcgagtcaagcattggtcttggcaaacagggactgagctgtaacctctgtaagtacactgttcacgaccagtgtgccatgaaagccctgccttgtgaagtcagcacctatgccaagtctcggaaggacattggtgtccaatcacatgtgtgggtgcgaggaggctgtgagtccgggcgctgcgaccgctgtcagaaaaagatccggatctaccacagtctgaccgggctgcattgtgtatggtgccacctagagatccacgatgactgcctgcaagcggtgggccatgagtgtgactgtgggctgctccgggatcacatcctgcctccatcttccatctatcccagtgtcctggcctctggaccggatcgtaaaaatagcaaaacaagccagaagaccatggatgatttaaatttgagcacctctgaggctctgcggattgaccctgttcctaacacccacccacttctcgtctttgtcaatcctaagagtggcgggaagcaggggcagagggtgctctggaagttccagtatatattaaaccctcgacaggtgttcaacctcctaaaggatggtcctgagatagggctccgattattcaaggatgttcctgatagccggattttggtgtgtggtggagacggcacagtaggctggattctagagaccattgacaaagctaacttgccagttttgcctcctgttgctgtgttgcccctgggtactggaaatgatctggctcgatgcctaagatggggaggaggttatgaaggacagaatctggcaaagatcctcaaggatttagagatgagtaaagtggtacatatggatcgatggtctgtggaggtgatacctcaacaaactgaagaaaaaagtgacccagtcccctttcaaatcatcaataactacttctctattggcgtggatgcctctattgctcatcgattccacatcatgcgagagaaatatccggagaagttcaacagcagaatgaagaacaagctatggtacttcgaatttgccacatctgaatccatcttctcaacatgcaaaaagctggaggagtctttgacagttgagatctgtgggaaaccgctggatctgagcaacctgtccctagaaggcatcgcagtgctaaacatccctagcatgcatggtggctccaacctctggggtgataccaggagaccccatggggatatctatgggatcaaccaggccttaggtgctacagctaaagtcatcaccgaccctgatatcctgaaaacctgtgtaccagacctaagtgacaagagactggaagtggttgggctggagggtgcaattgagatgggccaaatctataccaagctcaagaatgctggacgtcggctggccaagtgctctgagatcaccttccacaccacaaaaacccttcccatgcaaattgacggagaaccctggatgcagacgccctgtacaatcaagatcacccacaagaaccagatgcccatgctcatgggcccacccccccgctccaccaatttctttggcttcttgagcggatcctcggagacagtgcggtttcagggacaccaccaccatcaccactga

SEQ ID NO: 2 - protein, 751 aa, Homo sapiens
MAKERGLISPSDFAQLQKYMEYSTKKVSDVLKLFEDGEMAKYVQGDAIGYEGFQQFLKIYLEVDNVPRHLSLALFQSFETGHCLNETNVTKDVVCLNDVSCYFSLLEGGRPEDKLEFTFKLYDTDRNGILDSSEVDKIILQMMRVAEYLDWDVSELRPILQEMMKEIDYDGSGSVSQAEWVRAGATTVPLLVLLGLEMTLKDDGQHMWRPKRFPRPVYCNLCESSIGLGKQGLSCNLCKYTVHDQCAMKALPCEVSTYAKSRKDIGVQSHVWVRGGCESGRCDRCQKKIRIYHSLTGLHCVWCHLEIHDDCLQAVGHECDCGLLRDHILPPSSIYPSVLASGPDRKNSKTSQKTMDDLNLSTSEALRIDPVPNTHPLLVFVNPKSGGKQGQRVLWKFQYILNPRQVFNLLKDGPEIGLRLFKDVPDSRILVCGGDGTVGWILETIDKANLPVLPPVAVLPLGTGNDLARCLRWGGGYEGQNLAKILKDLEMSKVVHMDRWSVEVIPQQTEEKSDPVPFQIINNYFSIGVDASIAHRFHIMREKYPEKFNSRMKNKLWYFEFATSESIFSTCKKLEESLTVEICGKPLDLSNLSLEGIAVLNIPSMHGGSNLWGDTRRPHGDIYGINQALGATAKVITDPDILKTCVPDLSDKRLEVVGLEGAIEMGQIYTKLKNAGRRLAKCSEITFHTTKTLPMQIDGEPWMQTPCTIKITHKNQMPMLMGPPPRSTNFFGFLSGSSETVRFQGHHHHHH

SEQ ID NO: 3 - DNA, 2838 bp, Homo sapiens
  source 1..2838: mol_type = genomic DNA
atggagccgcgggacggtagccccgaggcccggagcagcgactccgagtcggcttccgcctcgtccagcggctccgagcgcgacgccggtcccgagccggacaaggcgccgcggcgactcaacaagcggcgcttcccggggctgcggctcttcgggcacaggaaagccatcacgaagtcgggcctccagcacctggccccccctccgcccacccctggggccccgtgcagcgagtcagagcggcagatccggagtacagtggactggagcgagtcagcgacatatggggagcacatctggttcgagaccaacgtgtccggggacttctgctacgttggggagcagtactgtgtagccaggatgctgcagaagtcagtgtctcgaagaaagtgcgcagcctgcaagattgtggtgcacacgccctgcatcgagcagctggagaagataaatttccgctgtaagccgtccttccgtgaatcaggctccaggaatgtccgcgagccaacctttgtacggcaccactgggtacacagacgacgccaggacggcaagtgtcggcactgtgggaagggattccagcagaagttcaccttccacagcaaggagattgtggccatcagctgctcgtggtgcaagcaggcataccacagcaaggtgtcctgcttcatgctgcagcagatcgaggagccgtgctcgctgggggtccacgcagccgtggtcatcccgcccacctggatcctccgcgcccggaggccccagaatactctgaaagcaagcaagaagaagaagagggcatccttcaagaggaagtccagcaagaaagggcctgaggagggccgctggagacccttcatcatcaggcccaccccctccccgctcatgaagcccctgctggtgtttgtgaaccccaagagtgggggcaaccagggtgcaaagatcatccagtctttcctctggtatctcaatccccgacaagtcttcgacctgagccagggagggcccaaggaggcgctggagatgtaccgcaaagtgcacaacctgcggatcctggcgtgcgggggcgacggcacggtgggctggatcctctccaccctggaccagctacgcctgaagccgccaccccctgttgccatcctgcccctgggtactggcaacgacttggcccgaaccctcaactggggtgggggctacacagatgagcctgtgtccaagatcctctcccacgtggaggaggggaacgtggtacagctggaccgctgggacctccacgctgagcccaaccccgaggcagggcctgaggaccgagatgaaggcgccaccgaccggttgcccctggatgtcttcaacaactacttcagcctgggctttgacgcccacgtcaccctggagttccacgagtctcgagaggccaacccagagaaattcaacagccgctttcggaataagatgttctacgccgggacagctttctctgacttcctgatgggcagctccaaggacctggccaagcacatccgagtggtgtgtgatggaatggacttgactcccaagatccaggacctgaaaccccagtgtgttgttttcctgaacatccccaggtactgtgcgggcaccatgccctggggccaccctggggagcaccacgactttgagccccagcggcatgacgacggctacctcgaggtcattggcttcaccatgacgtcgttggccgcgctgcaggtgggcggacacggcgagcggctgacgcagtgtcgcgaggtggtgctcaccacatccaaggccatcccggtgcaggtggatggcgagccctgcaagcttgcagcctcacgcatccgcatcgccctgcgcaaccaggccaccatggtgcagaaggccaagcggcggagcgccgcccccctgcacagcgaccagcagccggtgccagagcagttgcgcatccaggtgagtcgcgtcagcatgcacgactatgaggccctgcactacgacaaggagcagctcaaggaggcctctgtgccgctgggcactgtggtggtcccaggagacagtgacctagagctctgccgtgcccacattgagagactccagcaggagcccgatggtgctggagccaagtccccgacatgccagaaactgtcccccaagtggtgcttcctggacgccaccactgccagccgcttctacaggatcgaccgagcccaggagcacctcaactatgtgactgagatcgcacaggatgagatttatatcctggaccctgagctgctgggggcatcggcccggcctgacctcccaacccccacttcccctctccccacctcaccctgctcacccacgccccggtcactgcaaggggatgctgcaccccctcaaggtgaagagctgattgaggctgccaagaggaacgacttctgtaagctccaggagctgcaccgagctgggggcgacctcatgcaccgagacgagcagagtcgcacgctcctgcaccacgcagtcagcactggcagcaaggatgtggtccgctacctgctggaccacgcccccccagagatccttgatgcggtggaggaaaacggggagacctgtttgcaccaagcagcggccctgggccagcgcaccatctgccactacatcgtggaggccggggcctcgctcatgaagacagaccagcagggcgacactccccggcagcgggctgagaaggctcaggacaccgagctggccgcctacctggagaaccggcagcactaccagatgatccagcgggaggaccaggagacggctgtgggatcctcggagacagtgcggtttcagggacaccaccaccatcaccactga

SEQ ID NO: 4 - protein, 945 aa, Homo sapiens
MEPRDGSPEARSSDSESASASSSGSERDAGPEPDKAPRRLNKRRFPGLRLFGHRKAITKSGLQHLAPPPPTPGAPCSESERQIRSTVDWSESATYGEHIWFETNVSGDFCYVGEQYCVARMLQKSVSRRKCAACKIVVHTPCIEQLEKINFRCKPSFRESGSRNVREPTFVRHHWVHRRRQDGKCRHCGKGFQQKFTFHSKEIVAISCSWCKQAYHSKVSCFMLQQIEEPCSLGVHAAVVIPPTWILRARRPQNTLKASKKKKRASFKRKSSKKGPEEGRWRPFIIRPTPSPLMKPLLVFVNPKSGGNQGAKIIQSFLWYLNPRQVFDLSQGGPKEALEMYRKVHNLRILACGGDGTVGWILSTLDQLRLKPPPPVAILPLGTGNDLARTLNWGGGYTDEPVSKILSHVEEGNVVQLDRWDLHAEPNPEAGPEDRDEGATDRLPLDVFNNYFSLGFDAHVTLEFHESREANPEKFNSRFRNKMFYAGTAFSDFLMGSSKDLAKHIRVVCDGMDLTPKIQDLKPQCVVFLNIPRYCAGTMPWGHPGEHHDFEPQRHDDGYLEVIGFTMTSLAALQVGGHGERLTQCREVVLTTSKAIPVQVDGEPCKLAASRIRIALRNQATMVQKAKRRSAAPLHSDQQPVPEQLRIQVSRVSMHDYEALHYDKEQLKEASVPLGTVVVPGDSDLELCRAHIERLQQEPDGAGAKSPTCQKLSPKWCFLDATTASRFYRIDRAQEHLNYVTEIAQDEIYILDPELLGASARPDLPTPTSPLPTSPCSPTPRSLQGDAAPPQGEELIEAAKRNDFCKLQELHRAGGDLMHRDEQSRTLLHHAVSTGSKDVVRYLLDHAPPEILDAVEENGETCLHQAAALGQRTICHYIVEAGASLMKTDQQGDTPRQRAEKAQDTELAAYLENRQHYQMIQREDQETAVGSSETVRFQGHHHHHH

SEQ ID NO: 5 - DNA, 2214 bp, Homo sapiens
  source 1..2214: mol_type = other DNA
atggcttccccaagcgacttcgcccagctgcagaagtacatggaatacagcaccaagaaggtgtctgacgtcctgaagctgttcgaggacggtgaaatggctaagtacgtccagggcgacgctatcggatacgagggattccagcagttcctgaagatctacctggaagtggacaacgtccccaggcacctgtcactggctctgttccagtccttcgagactggccactgcctgaacgaaaccaacgtcactaaggacgtggtctgcctgaacgacgtgagctgctacttctctctgctggagggtggcagaccagaggacaagctggaattcaccttcaagctgtacgacactgaccgcaacggaatcctggactccagcgaagtggacaagatcatcctgcagatgatgcgtgtcgctgagtacctggactgggacgtgagcgaactgaggcctatcctgcaggagatgatgaaggaaatcgactacgacggctctggatcagtgtcccaggctgagtgggtccgcgctggtgctaccactgtgccactgctggtcctgctgggactggaaatgaccctgaaggacgacggtcagcacatgtggcgcccaaagcgtttccccaggccagtctactgcaacctgtgcgagtcttcaatcggtctgggcaagcagggcctgtcatgcaacctgtgcaagtacaccgtgcacgaccagtgcgctatgaaggccctgccctgcgaggtctcaacttacgctaagtcccgtaaggacatcggagtgcagtcacacgtgtgggtcaggggaggttgcgaatccggtagatgcgaccgctgccagaagaagatccgtatctaccactccctgaccggactgcactgcgtctggtgccacctggagatccacgacgactgcctgcaggccgtgggacacgaatgcgactgcggtctgctgcgtgaccacatcctgcctccctccagcatctacccttcagtcctggcttccggtcccgacaggaagaacagcaagacctctcagaagactatggacgacctgaacctgagcacctctgaggccctgcgcatcgaccctgtgcccaacactcacccactgctggtgttcgtcaaccctaagagcggcggaaagcagggtcagagagtcctgtggaagttccagtacatcctgaacccacgccaggtgttcaacctgctgaaggacggccctgagatcggactgagactgttcaaggacgtgcccgactctcgcatcctcgtctgcggtggcgacggtactgtgggatggatcctggaaactatcgacaaggctaacctgccagtgctgccacctgtggctgtcctgccactgggaaccggtaacgacctggctcgttgcctgcgttggggaggtggctacgagggacagaacctggccaagatcctgaaggacctggaaatgagcaaggtggtccacatggacagatggtctgtggaggtcatcccacagcagactgaggaaaagtcagacccagtccctttccagatcatcaacaactacttcagcatcggtgtggacgcttctatcgcccacagattccacatcatgcgcgagaagtaccctgaaaagttcaactcccgcatgaagaacaagctgtggtacttcgagttcgctacctcagaatccatcttctcaacttgcaagaagctggaggaatccctgaccgtcgagatctgcggcaagcctctggacctgtcaaacctgtccctggaaggcatcgctgtgctgaacatcccaagcatgcacggaggttctaacctctggggcgacactaggaggcctcacggtgacatctacggcatcaaccaggccctgggagctaccgccaaggtcatcactgaccccgacatcctgaagacctgcgtgccagacctgagcgacaagcgtctggaggtggtcggactggagggtgccatcgaaatgggccagatctacactaagctgaagaacgctggaaggagactggccaagtgctctgagatcaccttccacaccactaagactctgcctatgcagatcgacggtgaaccctggatgcagaccccatgcactatcaagatcacccacaagaaccagatgcccatgctgatgggtcctcctcctcgctctggatcttcagaaactgtgaggttccagggccaccaccaccaccaccactga

SEQ ID NO: 6 - protein, 737 aa, Homo sapiens
MASPSDFAQLQKYMEYSTKKVSDVLKLFEDGEMAKYVQGDAIGYEGFQQFLKIYLEVDNVPRHLSLALFQSFETGHCLNETNVTKDVVCLNDVSCYFSLLEGGRPEDKLEFTFKLYDTDRNGILDSSEVDKIILQMMRVAEYLDWDVSELRPILQEMMKEIDYDGSGSVSQAEWVRAGATTVPLLVLLGLEMTLKDDGQHMWRPKRFPRPVYCNLCESSIGLGKQGLSCNLCKYTVHDQCAMKALPCEVSTYAKSRKDIGVQSHVWVRGGCESGRCDRCQKKIRIYHSLTGLHCVWCHLEIHDDCLQAVGHECDCGLLRDHILPPSSIYPSVLASGPDRKNSKTSQKTMDDLNLSTSEALRIDPVPNTHPLLVFVNPKSGGKQGQRVLWKFQYILNPRQVFNLLKDGPEIGLRLFKDVPDSRILVCGGDGTVGWILETIDKANLPVLPPVAVLPLGTGNDLARCLRWGGGYEGQNLAKILKDLEMSKVVHMDRWSVEVIPQQTEEKSDPVPFQIINNYFSIGVDASIAHRFHIMREKYPEKFNSRMKNKLWYFEFATSESIFSTCKKLEESLTVEICGKPLDLSNLSLEGIAVLNIPSMHGGSNLWGDTRRPHGDIYGINQALGATAKVITDPDILKTCVPDLSDKRLEVVGLEGAIEMGQIYTKLKNAGRRLAKCSEITFHTTKTLPMQIDGEPWMQTPCTIKITHKNQMPMLMGPPPRSGSSETVRFQGHHHHHH